(19) United States
(12) Patent Application Publication
(10) Pub. No.: US 2008/0233049 A1
(43) Pub. Date: Sep. 25, 2008

(54) Agents And Methods For Early Diagnosis And Monitoring Of Alzheimer's Disease And Other Neurological Disorders

(75) Inventors: Hiroaki Tani, Redwood City, CA (US); Ian Andrew Ferguson, Queensland (AU)

Correspondence Address: KNOBBE MARTENS OLSON & BEAR LLP, 2040 MAIN STREET, FOURTEENTH FLOOR, IRVINE, CA, 92614, US

(73) Assignee: Ian Andrew Ferguson, Crafers West (AU)

(21) Appl. No.: 11/571,241

(22) PCT Filed: Jun. 23, 2005
(86) PCT No.: PCT/IB05/04077

§ 371 Date: Nov. 23, 2007

(30) Foreign Application Priority Data
Jun. 23, 2004 (AU) 2004/903380

(51) Int. Cl. A61K 49/00; A61K 49/04

(52) U.S. Cl. 424/9.4; 424/9.1; 424/9.6

(57) ABSTRACT

This invention is in the fields of medicine and neurology, and relates to methods and agents for early diagnosis and monitoring of Alzheimer's disease and other neurological disorders. More particularly, the present invention provides the method for evaluating the health of central nervous system neurons in a human patient, comprising the steps of: a) administering to said patient a population of molecular complexes comprising (i) a polypeptide capable of activating neuronal endocytosis, axonal transport, and synaptic transfer; and (ii) an imaging agent suited for determining location and evaluating neuronal transport of said molecular complexes within said patient; and b) using at least one imaging method to determine location and evaluate neuronal transport of said molecular complexes within said patient.

### BACKGROUND OF THE INVENTION

This invention is in the fields of medicine and neurology, and relates to methods and agents for early diagnosis and monitoring of Alzheimer's disease and other neurological disorders.

The pathological processes of Alzheimer's disease (AD) have been described in many books and review articles, including, Burggren and Bookheimer 2002, Martin 1999, and Selkoe 1997. In particular, beta-amyloid plaques (referred to herein simply as amyloid plaques, for convenience) are a defining trait of AD, and are inevitably found in the brains of elderly people who died while suffering from AD. However, the opinions of experts and researchers are divided over whether amyloid plaques are an active causative factor in AD, or are merely a symptom of and a response to other causative agents and stresses. This ongoing debate is presented in articles such as Walsh and Selkoe 2004 and Wevers et al 2002.

It also should be recognized that most advocates on either side in such debates do not take absolute positions, claiming that a single type of process can explain all cases of AD among all patients. Instead, various lines of evidence suggest that among different classes of AD patients, different originating factors (which may include, for example, capillary leakage, excessive activation of certain types of neurotransmitter receptors, inadequate activation of other types of neurotransmitter receptors, etc.) may contribute in different ways and at different levels to the damage processes in various patients. Accordingly, the formation and growth of amyloid plaques, in an aging person whose brain has one or more parts or systems that are effectively wearing out and losing the ability to function vigorously, may be a “convergent” type of response to various different triggering factors.

It also should be recognized that when significant numbers of amyloid plaques have begun forming in the brain of an aging person, they apparently can accelerate and aggravate the progression of subsequent damage, by means that include the formation of destructive oxidative radicals within the amyloid plaques, catalyzed by copper ions. Accordingly, when an aging person begins forming significant numbers of amyloid plaques in the brain, either as a natural aging process or triggered by one or more causative factors that likely will never be identified, the initial amyloid plaques may begin inflicting more damage on the brain, through radical-mediated and possibly other processes. This will lead to more stress, and to the formation and growth of more amyloid plaques, in a “vicious circle” type of pathology. This aspect of the problem, in which initial amyloid plaques that may have been triggered by some other factor begin to accelerate the progression and severity of the damage and disease, has rendered it exceptionally difficult and effectively impossible for researchers to clearly sort out and identify the precise roles that amyloid plaques play, in different AD patients.

There are powerful factors and incentives that are driving numerous research teams to try to find better ways to perform early diagnosis of AD, and of neurological conditions referred to by terms such as pre-Alzheimer's, mild cognitive impairment, etc. One powerful set of motivating factors centers around the hope that if people can be diagnosed early, steps can be taken to slow the progression of the disease, before the damage becomes serious or severe. Such steps might include, for example, administering drugs (or combinations thereof) that can exert various neuroactive effects that may be able to help at least some cases of Alzheimer's disease. Examples of such drugs that are in human clinical trials, or that are already available for sale, include donepezil (sold under the trademark ARICEPT), galantamine (REMINYL), and rivastigmine (EXELON), which increase the levels of acetylcholine, an excitatory neurotransmitter, in aging brains; memantine (NAMENDA), a drug that slows down excitatory activity at the NMDA class of glutamate receptors; Neurochem's NC-531 (ALZHEMED), a compound that slows down amyloid plaque formation; and flurbiprofen (FLURIZAN), a non-steroidal anti-inflammatory drug that helps suppress various cellular responses to minor triggering events.

Another line of research has indicated that chelation (i.e., sequestering and inactivation) of zinc and copper, by a drug such as clioquinol, may help slow or even reverse the growth of amyloid plaques, thereby reducing or preventing any damage processes that may be caused or aggravated by such plaques (e.g., Bush 2002).

Still other research, including work described in published PCT patent application WO 2003/091387 (by the same inventors herein, and discussed in more detail below) and in various patents and articles by W. H. Frey (including Frey 2002, and U.S. Pat. Nos. 5,624,898; 6,313,093; 6,342,478; and 6,407,061), indicate that researchers are developing effective ways to transport polypeptides across the blood brain barrier. If “neurotrophic” hormones such as nerve growth factor (NGF) are delivered into brain tissue at appropriate locations, they may offer profound, lasting, and benefits in actually reversing the damage caused by AD or other neurodegenerative diseases.

Any or all of those types of therapies can be vastly enhanced and improved, by diagnosing and monitoring AD while it is still in the early or very early stages. The presumption is that once a neuron has died, it is dead and gone, forever, and can never be replaced. Accordingly, if problems that are causing neurological stress and damage can be detected and treated, before the neurons are pushed and damaged to a point where they are doomed, then such treatments that begin at an early stage have a much better chance of offering real and substantial benefits. As a result, efforts to diagnose AD while it is still at an early stage have become very important, and are described in articles such as Reed and Janust 1999, DeKosky and Marek 2003, Klunk et al 2003, and Mathis et al 2005.

It should also be noted that effective and reliable early diagnosis and monitoring can do enormous good, by providing researchers and patients with better and more reliable ways to monitor and measure cellular and biochemical indicators within a span of weeks or months, rather than having to wait for years and then having to interpret subjective results (such as performance on cognitive or memory tests) that can be seriously affected by factors such as how well or how poorly an elderly patient happens to be feeling on the day of the test.

### Axons, Axonal Transport, and Axonopathy

Several types of neuronal structures and processes need to be briefly summarized, since they are important in this invention.

One set of important terms centers around the words axon, axonal, and axonopathy. The axon is the longest and largest fiber-like (or finger-like) projection (also called a process, dendrite, etc.) that emerges from the main cell body of a neuron (the main body is the portion of the neuron that contains the nucleus, along with various other components and organelles). Numerous other smaller fibers (or processes) typically branch off from the axon; nevertheless, the axon can be clearly identified, because it is the longest and largest extension of the cell.

Axonal transport of nutrients and various other molecules is very important, in CNS neurons, and it can proceed in either direction. “Retrograde” transport includes transport of any type of molecule (such as a nutrient, hormone, etc.) from a “distal” location on an axon, toward the main body and nucleus of the neuron; in layman's terms, retrograde transport travels in an “inward” direction, toward the center of the cell. The other direction is called “anterograde” transport, and includes transport of any molecule (such as a protein molecule that was synthesized in the main body of the cell), toward a distal location on the axon (in layman's terms, in an “outward” direction, away from the center of the cell).

Both types of transport involves scaffold-type structures known as “microtubules”, which function in a manner comparable to rails. Specialized transport proteins (often called “motor proteins”, including a class of proteins called “kinesin” proteins, derived from the same word as “kinetic”, which refers to motion) grip the microtubules, and can travel along the microtubule “rails” while effectively towing or pushing various types of molecules, often referred to as passenger, cargo, freight, or payload molecules.

These structures and activities, and adverse conditions called “axonopathy”, are discussed in articles such as Stokin et al 2005, which reported that in rodent models of Alzheimer's disease, and in limited confirmatory tests in humans, certain types of axonal defects and cellular transport problems began to occur at least a year before the formation of amyloid plaques. Specialized strains of mice with certain types of knockout genes or other genetic defects (created by genetic engineering, selective breeding, or other methods) have defective microtubule and/or transport proteins, which impair or block their ability to transport various molecules that need to be transported within neuronal axons. For example, Stokin et al used mice that were engineered to suffer from “knockout” mutations in the gene that encodes the “kinesin-1 light chain” (KLC) portion of the kinesin transport protein.

Wild-type mice and rats do not form amyloid plaques in their brains, and do not suffer from age-related syndromes that are regarded as models of AD. However, because of the huge worldwide importance of AD, it has been the focus of huge amounts of research effort, and numerous teams have created genetically engineered strains of mice and rats with foreign genes that cause the formation of amyloid or amyloid-like plaques. For example, the main mouse strain used by Stokin et al, designated as the Tg-swAPP^{Prp} strain, carries genes that directly encode the human version of beta-amyloid proteins, under the control of strong promoters. Rather than waiting for a slow and gradual accumulation of small quantities of beta-amyloid proteins, as occur in humans because of low rates of improper handling of amyloid precursor proteins, the human amyloid genes inserted into mice can create amyloid plaques at much faster rates.

Accordingly, Stokin et al studied Tg-swAPP^{Prp} mice strains (which form human amyloid deposits in their brains) that were engineered to also suffer from KLC gene defects (which impaired their ability to carry out axonal transport). The results of those studies indicated that those animals initially accumulated symptoms and indicators of axonopathy and transport defects, within the neuronal axons in their brains, which were followed, a year or more later, by the development of Alzheimer-type symptoms.

Although questions arise about how accurately that type of doubly-impaired mouse model can accurately model Alzheimer;s disease in humans, that report tends to support the argument that amyloid plaques, in at least some AD patients, are likely to arise as a result of other triggering factors, rather than being the initiating cause of AD.

This current invention also rests on the belief and growing evidence that in at least some and probably most cases of AD, a patient will begin suffering from one or more types of neurodegenerative processes that will precede and predate amyloid plaque formation, by months or even years. Accordingly, the challenge is to establish and develop a diagnostic and analytic method that can identify and quantify one or more indicators of one or more types of neurodegenerative processes that, if uncorrected, will eventually lead to amyloid plaque formation and the development of “classical” Alzheimer's disease.

In addition, neuronal transport defects (including axonopathy problems) are also likely to be involved in at least some other types neurodegenerative diseases, such as amyotrophic lateral sclerosis (ALS), Parkinson's disease, etc. As with Alzheimer's disease, no experts in this field would assert that any single type of initial causative (etiological) factor can explain any and all cases of ALS, Parkinson's disease, or other neurodegenerative diseases. Instead, neurodegenerative diseases usually are diagnosed based on the types of cells that are being damaged and destroyed. As a result, any of various different processes that damage a certain class of cells, in the brain or spinal cord, can lead to the neurological disorder that is associated with impairments and degeneration in that particular class of cells, in the brain or spinal cord.

To properly set the stage for the invention disclosed herein, another line of technology needs to be described.

### Screening and Selection of Endocytotic Polypeptides

Two scientific and medical developments of potentially major importance, in both research and medicine, have been created by the inventors herein. Both developments are described in detail in a published PCT application, WO 2003/091387 (a full copy can be downloaded for a minimal charge from sources such as www.delphion.com). Those two developments are briefly summarized herein, because they both play an essential role in this invention, which builds upon, extends, and expands the teachings in that prior application.

The first major development disclosed in WO 2003/091387 describes methods and agents for delivering polypeptides through the blood-brain barrier (BBB), into specific and targeted regions of brain or spinal tissue. This is a breakthrough of major importance, because most of the important CNS hormones are polypeptides (such as nerve growth factor) rather than non-peptide molecules (such as adrenaline). There is an important reason why the brain evolved with peptide rather than non-peptide hormones and other regulating factors. Cells and tissues can exert tight and more reliable control over polypeptides, which are expressed directly by genes, by using any of numerous methods for regulating gene expression. By contrast, non-peptide molecules (such as adrenaline) are created by enzymes, and it is very difficult for cells and tissues to carefully regulate the amounts of small molecules made by enzymes, once the enzymes have been created and are active.

That new method for delivering polypeptides into targeted regions of brain or spinal tissue can be summarized as follows:

(1) a genetic vector is used to transfect one or more neurons that “straddle” the BBB, such as sensory neurons, nocioceptive neurons, or lower motor neurons. This is done by administering a liquid that contains millions of copies of the genetic vector (which can use and exploit the “infection and delivery” systems of disarmed viruses, or similar mechanisms), at a targeted location that causes the vectors to contact and enter neuronal projections that are accessible, outside the BBB;

(2) after the vector (or a portion thereof, such as the DNA carried by the vector) has entered the accessible tip or fiber of a BBB-straddling neuron, the vector (or a portion thereof) will be transported to the main cell body of the neuron, using the natural process called retrograde transport;

(3) once inside the main cell body, genes carried by the genetic vectors can be expressed, to form polypeptides, by using and exploiting the types of invasive and infective mechanisms that function in viruses;

(4) at least some of the polypeptides that are expressed from the genes carried by the vector will be transported, by the neurons, to secretion sites that are located fully inside the BBB. This is done by anterograde transport, and it can be increased (if necessary) by using genetically-engineered gene sequences (carried by the vector) to encode a “leader” or “transport” polypeptide sequence, as part of the polypeptide that is expressed by the gene carried by the vector;

(5) at least some of the polypeptides that are transported to the secretion sites inside the BBB will be secreted, by the transfected neurons. Those secreted polypeptides will then contact and exert their effects upon secondary “target” neurons that are located entirely within the BBB.

In other words, certain specific types of neurons that “straddle” the BBB are used as active conduits, to provide passageways through the BBB. The tips of the BBB-straddling neurons, which are accessible outside the BBB, are contacted and infected with genetic vectors of a “disarmed virus” type (or certain other classes of vectors that can accomplish similar results). Using mechanisms comparable to a viral infection, the genes carried by the foreign vectors induce the transfected host cells to make the polypeptides encoded by the vectors. Then, using natural cellular mechanisms, the neuron will transport the vector-encoded polypeptides deeper into the brain or spine, and release the polypeptides inside BBB-protected brain or spinal tissue, in specific targeted “secondary” brain or spinal regions that are known to interact with the BBB-straddling neurons that provided the conduits.

Accordingly, by altering and exploiting several mechanisms that were initially found in certain specialized viruses that can infect neurons, it becomes possible to deliver useful and therapeutic polypeptides (such as nerve growth factor and other neurotrophic hormones) into the brain of an elderly person who otherwise faces an irreversible slide into a neurodegenerative disease, or into the spinal cord of a person who has suffered an injury that otherwise might lead to lifelong paralysis and suffering.

That was not the only major disclosure in PCT application WO 2003/091387. A second major disclosure was provided in that application, because it provides certain crucially important elements of the system. That second disclosure involves in vivo screening and selection techniques that can be used to identify and isolate polypeptide sequences (i.e., amino acid sequences within polypeptide segments) that can function in a manner referred to herein as “locomotive” polypeptides. They are given that descriptive name, because they can function in a manner analogous to locomotives, which can pull freight cars that have been loaded with any type of freight (or passengers, baggage, “payload”, etc.) that an operator chooses to load into the freight (or passenger) cars.

Briefly, this in vivo screening method involves injecting, into lab animals such as rats, a library of specialized phages (i.e., special types of relatively small viruses, which have been modified and engineered in ways that make them extremely useful in laboratory work that can move back and forth between bacterial cell hosts, and mammalian cell hosts). Billions of different phages can be contained in each library, and highly sophisticated libraries are available from companies that have gone to great lengths to ensure enormous diversity and range in their libraries. Each phage carried a certain relatively small polypeptide segment, inserted near the middle of a coat protein that is assembled into the coat the surrounds the virus.

A liquid carrying the phage library (or some portion of the library) is injected into a rat, into a location where the phages will contact receptors on the surfaces of neurons that have relatively long fibers (such as the sciatic nerve bundle, a nerve bundle that travels from the hip down to the foot). Over the next few hours, out of the millions or billions of candidates in the library, those particular phages that happen to contain polypeptide sequences that have “endocytotic” activity will be taken inside the neuronal fibers. The terms endocytotic and endocytosis refer to a process of transporting something inside a cell; this process usually involves specialized cell surface receptors, which are described in nearly any textbook on cell biology.

Some but not all of the phages that are taken inside long neuronal fibers will be transported, within the neuronal fibers, to a location that is some distance away from the site of injection. If the injection site is near the foot or knee of a rat, that transport will be in a retrograde direction, since the main cell bodies of sciatic nerves are near the hip, closer to the spinal cord.

After a suitable period of time (such as 18 hours) has passed, the rats are sacrificed, and at a specific “harvesting” site, neuronal segments are harvested, to gather any phages that entered the neurons and were transported to the harvesting site. This harvesting process can be enhanced, to provide high concentrations of phages at a specific location, by tightening a loop of suture material around the sciatic nerve bundle at the harvesting site, in a way that impedes the flow and transport of fluids (and virus particles) across the constriction that is created by the tight loop of suture material. The harvested nerve segments are homogenized in a way that releases the phage particles without damaging them, and the phages are isolated and reproduced, in culture dishes.

Because the harvesting site is located several millimeters away from the injection site, the only phages that will be present, in the harvested nerve segments, are phages that were indeed taken into the nerve cell fibers, and transported in a retrograde direction within those fibers. The amino acid sequences of several phages that were shown to be taken into and transported by sciatic nerve bundles, in these types of tests, are listed in Tables 1-3.

TABLE 1
In vivo selection of 1^{st} round in vitro anti-p75^{NTR} population

| Seq. | Partial CDR | ELISA | | % total | Expansion | ELISA | Internalsation | Expansion |
|---|---|---|---|---|---|---|---|---|
| ID | Sequence | Score | % total | Input | Score | Rank | Rank | Rank |
| H | SYWIG-IVS | 1.38 | 1.04 | 0.69 | 1.50 | 1 | 6 | 4 |
| F | SGTYYWS-RIY | 1.18 | 3.65 | 0.69 | 5.25 | 2 | 2 | |
| L | SYWIN-HIY | 1.08 | 3.65 | 3.47 | 1.05 | 3 | 2 | 5 |
| D | NYAMT-TIS | 1.02 | 1.56 | 0.69 | 2.25 | 4 | 5 | 3 |
| K | NSHYYWA-YIY | 0.93 | 1.56 | 0.69 | 2.25 | 5 | 5 | 3 |
| B | SYGMH-VIS-- | 0.92 | 5.21 | 5.56 | 0.94 | 6 | 1 | 6 |
| | QGDS | | | | | | | |
| M | DYAHH-GIS | 0.90 | 1.56 | 0.69 | 2.25 | 7 | 5 | 3 |
| E | SGYHWG-AIY | 0.89 | 1.56 | 0.69 | 2.25 | 8 | 5 | 3 |
| A | SYGMH-VIS-- | 0.87 | 3.13 | 1.39 | 2.25 | 9 | 3 | 3 |
| | RASQ | | | | | | | |
| N | GYGVN-MIW- | 0.86 | 2.08 | 0.69 | 3.00 | 10 | 4 | 2 |
| | DY | | | | | | | |
| I | NYPIS-GII | 0.85 | 1.04 | 0.69 | 1.50 | 11 | 6 | 4 |
| G | ELSMH-GFD | 0.84 | 1.04 | 0.69 | 1.50 | 12 | 6 | 4 |
| Q | SYAIS-GII-DI | 0.84 | 1.56 | 0.69 | 2.25 | 13 | 5 | 3 |
| J | RHEMT-YIS | 0.81 | 2.08 | 1.39 | 1.50 | 14 | 4 | 4 |
| C | SYGMH-VIS-- | 0.77 | 1.04 | 0.69 | 1.50 | 15 | 6 | 4 |
| | TGTS | | | | | | | |
| P | SYYWN-YIY | 0.75 | 1.04 | 0.69 | 1.50 | 16 | 6 | 4 |
| R | DYYMS-HIS | 0.71 | 1.56 | 0.69 | 2.25 | 17 | 5 | 3 |
| O | DHIMN-RIY | 0.62 | 2.08 | 0.69 | 3.00 | 18 | 4 | 2 |

TABLE 2
In vitro selection against p75^{NTR}

| | | | | |
|---|---|---|---|---|
| | Partial | Percentage | ELISA | |
| Sequence ID | CDR sequence | Occurrence | score | Internalising? |

1^{st} round in vitro

| | | | | |
|---|---|---|---|---|
| T | NYAMH-WIH | 10.42 | 0.83 | N |
| V | SYYWS-YIY | 7.64 | 0.67 | N |
| B | SYGMH-VIS-- | 5.56 | 0.75 | Y |
| | QGDS | | | |
| L | SYWIN-HIY | 3.47 | 0.72 | Y |
| U | GYYWS-VIS | 2.08 | 6.45 | N |
| S | SYGVS-WIN | 1.39 | 0.88 | N |
| J | RHEMT-YIS | 1.39 | 0.7 | Y |
| A | SYGMH-VIS-- | 1.39 | 0.76 | N |
| | RASQ | | | |

2^{nd} round in vitro

| | | | | |
|---|---|---|---|---|
| W | SYAIS-GII--DV | 12.5 | 4.04 | N |
| Q | SYAIS-GII-DI | 12.5 | 0.88 | Y |
| X | SNYWS-YIS | 6.25 | 3.63 | N |
| Z | TGGFSWG-FIY | 6.25 | 1.53 | N |
| Y | GYYWS-EIN | 4.17 | 0.9 | N |

3^{rd} round in vitro

| | | | | |
|---|---|---|---|---|
| Z | TGGFSWG-FIY | 21.88 | 2.34 | N |
| AB | GYYWS-DIK | 16.67 | 3.26 | N |
| Y | GYYWS-EIN | 7.29 | 5.74 | N |
| X | SNYWS-YIS | 6.25 | 6.33 | N |
| W | SYAIS-GII--DV | 5.21 | 4.34 | N |
| AE | SYAMS-AIS | 2.08 | 4.36 | N |
| AF | SYSWS-EIN | 2.08 | 11.64 | N |
| AG | NYVMY-AIS | 2.08 | 1.86 | N |

TABLE 3
In vivo selection of a fully diverse parental library

| Sequence | Partial | Percentage | ELISA | |
|---|---|---|---|---|
| ID | CDR sequence | Occurrence | score | Anti-p75^{NTR}? |
| B | SYGMH-VIS-- | 6.25 | 0.81 | Y |
| | QGDS | | | |
| K | NSHYYWA-YIY | 4.17 | 0.87 | Y |
| N | GYGVN-MIW-DY | 4.17 | 0.65 | Y |
| V | SYYWS-YIY | 3.13 | 0.85 | Y |
| AI | SYWIG-IVS | 2.08 | 1.24 | ? |
| F | SGTYYWS-RIY | 2.08 | 0.79 | Y |
| AN | SYSMN-YIS | 2.08 | 0.71 | ? |
| AO | TYAIS-GII | 2.08 | 0.78 | ? |
| AP | NSYIS-WIN | 2.08 | 0.79 | ? |
| AR | GYGVN-MIW-TT | 2.08 | 0.88 | ? |

That type of in vivo screening operation can be repeated any number of times, using the “best performing phages” from each screening cycle as the starting population for the next screening cycle.

When the screening process is deemed to be complete, the best-performing phages are isolated, and the amino acid sequences carried in the coat proteins of those phages are determined. Short polypeptide fragments containing those amino acid sequences, which can drive endocytosis and neuronal transport, can be created by chemical synthesis, fermentation, or any other suitable method. Those polypeptide segments can then function in a manner analogous to locomotives, which can pull freight cars loaded with any type of freight an operator loads into the freight cars.

The in vivo screening techniques described in PCT application WO 2003/091387 focused on sciatic nerves. That work has now been extended in the manner described below, to show that similar steps also work when phage libraries are administered to olfactory receptor neurons, by means of liquids that are sprayed into the nasal sinuses. Those in vivo screening tests showed that certain phages, carrying specific polypeptide sequences, were not only taken into and transported within the olfactory neuron fibers; in addition, they were also transferred to other neurons, including cholinergic neurons that are fully enclosed within the blood-brain barrier. None of those phages have been sequenced, to determine the amino acid sequences of the “locomotive” polypeptides, so that sequence information is not available, and is not included herein.

Nevertheless, polypeptide segments that have been selected by this type of in vivo screening process can function effectively as “locomotive” polypeptides. They can pull molecules through BBB-straddling neurons, until the molecules reach other neurons that are fully enclosed within, and protected by, the blood-brain barrier.

Accordingly, one object of this invention is to disclose methods and agents for identifying, measuring, and quantifying various types of neurodegenerative processes (including processes that, if uncorrected, will eventually lead to amyloid plaque formation and the development of Alzheimer's disease).

Another object of this invention is to disclose methods and agents for measuring the rates of axonal transport, and/or synaptic transfers, involving specific classes of BBB-straddling neurons and CNS neurons, since both axonal transport rates and synaptic transfer rates can provide highly useful information to allow researchers to assess the status, activity levels, and health of such BBB-straddling and CNS neurons.

Another object of this invention is to disclose methods and agents to identify, measure, and quantify the loss or impairment, by or among certain classes of neurons, of their ability to transport certain types of molecules within their axons or other fibers, and their ability to secrete or receive certain types of molecules, at their synaptic junctions, in a manner that can help physicians diagnose and monitor various types of neurodegenerative disorders, including early and very early stage Alzheimer's disease, amyotrophilc lateral sclerosis, Parkinson's disease, etc.

Another object of this invention is to provide researchers with better tools to evaluate the performance of candidate drugs that may be able to slow down or otherwise treat or prevent various neurodegenerative disorders, in both animal tests, and human clinical trials.

These and other objects of the invention will become more apparent through the following summary, drawings, and detailed description.

### SUMMARY OF THE INVENTION

Methods and agents are disclosed for measuring the rates of certain types of cellular activities involving brain or spinal neurons. These activity levels decrease in the early stages of certain types of neurodegenerative disorders, including Alzheimer's disease, Parkinson's disease, and amyotrophic laterial sclerosis. Therefore, such measurements using these methods and agents can enable early diagnosis and monitoring of such disorders, leading to earlier and more effective treatments.

The relevant activities involve: (i) transport of certain types of molecules through the axons of certain types of neurons that straddles the blood-brain barrier (BBB); and, (ii) transfer of such molecules, from BBB-straddling neurons, to other types of CNS neurons located entirely within the brain or spinal cord.

Accordingly, this method uses a liquid that contains specialized types of “transport and track” complexes, comprising: (i) polypeptide segments that have been selected for endocytotic activity and axonal transport, by means of in vivo selection and screening methods, and (ii) imaging molecules that can be tracked and followed by suitable imaging techniques, such as fluorescent photomicrographs in animal tests, or single photon emission computerized tomography (SPECT) imaging in human patients.

The complexes, suspended in a liquid, are administered to certain types of tissues in a manner that enables the complexes to be taken into the accessible fibrous projections of targeted BBB-straddling neurons. As one example, this can be done by using nasal sprays to contact the labelled complexes with the tips of olfactory receptor neurons. The complexes will enter the neuronal fibers via endocytotic surface receptors, and they will be transported through the neuronal fibers, first by retrograde and then by anterograde transport mechanisms, which occur naturally.

If properly screened and selected, the polypeptides (referred to herein as “locomotive” polypeptides) will be secreted into BBB-protected synaptic junctions, by the BBB-straddling neurons. They will then be taken up by other neurons that are fully protected within the BBB, in “synapse jumping” transfers. Continuing the example above, olfactory neurons will transfer certain types of molecules to cholinergic neurons in the basal forebrain, via synaptic transfers.

Since rates of axonal transport activities and synaptic transfer activities provide useful indicators of the health, status, and vigor of the neurons involved, the ability to follow and monitor both of those activities, using the “transport and track” complexes described herein, enables new types of medical research and diagnosis.

Similar types of molecular complexes, which use selected “locomotive” polypeptides, can carry therapeutic drugs (including neurotrophic hormones, if desired) rather than imaging molecules into BBB-protected tissues, for therapy or prevention of neurological disorders. This concept, and methods for screening, identifying, and isolating effective “locomotive” polypeptides, was previously disclosed in PCT patent application WO 2003/091387. Accordingly, the disclosures herein extend those discoveries into the new and additional field of early-stage diagnosis of neurodegenerative diseases.

It also was recognized, during the research that led to this invention, that certain types of molecular complexes that actively enter “nasal-associated lymphoid tissue” (NALT), which forms part of a mammalian immune system, can provide effective and useful adjuvants, which can help increase the efficacy and safety of vaccines that are administered by nasal sprays rather than needles.

### BRIEF DESCRIPTION OF THE DRAWINGS

FIG. 1 is a fluorescent photomicrograph showing phages that entered olfactory receptor neurons, were transported through the axons of the olfactory receptor neurons, and were then transferred into olfactory bulb tissues inside the blood-brain barrier, in mice, five minutes after administration via a nasal infusion.

FIG. 2 is a fluorescent photomicrograph showing phages that were transported deeped into the olfactory bulb, at 30 hours after nasal infusion.

FIG. 3 is a fluorescent photomicrograph showing phages that were transported into additional brain regions, including cortex regions and the hippocampus.

FIG. 4 depicts a molecular complex showing a “locomotive polypeptide” that can drive endytotic uptake and transport into CNS neurons, and a drug-binding polypeptide that can hold and then release a therapeutic drug.

FIG. 5 is a photomicrograph showing labelled phages that were transported into immune tissues in the nasal region.

FIG. 6 is a photomicrograph showing labelled phages that were transported into immune tissues in lymph nodes and antigen-presenting cells.

FIG. 7 is a depiction of a phage particles that carries a “locomotive polypeptide” as well as additional adjuvants that can stimulate an immune response, for improved vaccines that can be administered via oral or nasal sprays.

### DETAILED DESCRIPTION

As summarized above, specialized molecular complexes are disclosed herein, which are referred to as “transport and track” complexes, since they are designed to enable certain types of transport, within CNS neurons, while providing researchers and clinicians with the ability to follow and track their movements and locations within such neurons.

The transport functions of these complexes are provided by “locomotive” polypeptides, selected by in vivo screening as described in the Background section, above, and in PCT application WO 2003/091387.

The tracking functions are provided by means such as relatively simple and inexpensive fluorescent labeling, in tests involving mice, rats, or other animals that can be sacrificed for direct viewing or photography of tissue slices, samples, or extracts. For non-invasive imaging, as required by human research and medicine, more sophisticated tracking means must be used, such as (for example) using an isotope of iodine such as ¹²³I, which has a half-life of about 13 hours, and which can be imaged by a specialized type of positron emission tomography called single photon emission computerised tomography (SPECT). Both of those two tracking modes are merely exemplary, and any other tracking, imaging, or similar mode of detection that is suited for some particular type of test or treatment can be used, as will be recognized by those skilled in the art.

The types of molecular “transport and track” complexes disclosed herein are useful for analyzing two (or even three) types of neuronal activities that are highly important, in certain classes of CNS neurons. Since these neuronal activities have altered (usually decreased) levels of activity in various types of neurodegenerative disorders (including Alzheimer's disease, Parkinson's disease, and amyotrophic laterial sclerosis), use of “transport and track” complexes as disclosed herein can enable diagnosis and monitoring of such disorders, including early (and even very early) diagnosis of at least some disorders, believed to include Alzheimer's disease. This can help enable earlier diagnosis and treatment, which is exceptionally useful in neurodegenerative disorders, since early or very early treatment can help preserve and protect neurons and neuronal networks before they begin to suffer from irreversible damage.

For example, in the very early stages of Alzheimer's disease, there is a drop-off in the rates and frequencies of synaptic transfers between olfactory receptor neurons, and cholinergic neurons in the basal forebrain. It is believed that in many and perhaps most cases of Alzheimer's disease, decreased synaptic transfer rates are likely to commence months or even years before substantial quantities of amyloid plaques begin to appear in the brain of a victim. Therefore, measurement of synaptic transfer rates between BBB-straddling olfactory receptor neurons, and BBB-protected cholinergic neurons in the basal forebrain, can provide an early warning system for identifying people who are at elevated risk of Alzheimer's disease. Such patients can begin therapy at a very early stage, while they still retain essentially normal, not-yet-degraded cognitive and thinking capabilities, before later-stage processes (including amyloid plaque formation and growth) begin wreaking havoc in their brains.

In a similar manner, synaptic transfers between other classes of neurons are believed capable of providing early indicators of Parkinson's disease, and amyotrophic laterial sclerosis. In addition, other neurodegenerative diseases are likely to be added to these known classes, after the tools and methods herein have been disclosed and made available to neurology researchers.

In one preferred embodiment that focuses on diagnosis of early Alzheimer's disease, the methods and agents of this invention will utilize protein sequences that have been identified and selected by successive rounds of screening, using in vivo screening and selection techniques described in the examples below, supplemented by additional discussion in published PCT application WO 2003/091387.

Briefly, the screening method that was used herein, as disclosed in Example 1, using a single round of sciatic screening, followed by a single round of olfactory screening. A phage display library, containing huge numbers of phages carrying essentially random polypeptide segments, exposed on the surfaces of their coat proteins, was emplaced near the knee of an animal, packed in a gel around the end of severed sciatic nerve bundle. Over the next 18 hours, phages carrying polypeptide sequences that could trigger and activate endocytotic receptors on the sciatic nerve fibers were be taken into the fibers, and transported within the nerve fibers, toward the hip. However, a tightened suture strand placed around the sciatic nerve bundle, next to the hip, acted as a dam, or blockage, which prevented any transport of particles inside the fibers, past the dam. This constriction created a “harvesting site”, and short nerve fiber segments were removed from the harvesting site, after the animals were sacrificed. Phages that had indeed been transported to the harvesting site were isolated and reproduced. The amino acid sequences of the “locomotive” polypeptides in some of those particular phages are listed in Table 1.

That type of screening cycle can be repeated any number of times, using the “best performing phages” from one screening round (or cycle) as the starting population for additional screening. When the screening process is deemed complete, the amino acid sequences in the polypeptides carried by the best-performing phages can be determined. Polypeptide segments containing those amino acid sequences, which can drive both endocytosis and neuronal transport, can be created by chemical synthesis, fermentation, or any other suitable method.

Those polypeptide segments are referred to herein as “locomotive” polypeptides, since they are analogous to the locomotive that can pull freight or passenger cars that are loaded with any type of freight, passenger, or other “payloads” molecules that an operator chooses to attach to the locomotives.

The in vivo screening techniques described in PCT application WO 2003/091387 focused mainly on sciatic nerves in mice or rats, and on a particular receptor type known as the p75 receptor, which is “upregulated” (i.e., its expression is increased, leading to larger numbers of receptors on the surfaces of the neuronal fibers) if a neuron is injured or stressed. A number of phages that were indeed taken into (and transported by) mouse or rat sciatic fibers had their “passenger” polypeptides sequences, and the sequencing data (which is correlated with the numbers of phages that were detected, in the analyzed phage populations) are provided in Table 1. As described in Example 1, those screened and enriched phage populations were used as the starting populations, for the nasal screening cycle described in Example 1.

The sequence data for those phage populations are the only sequence data that are currently available, as this application is being filed. None of the “synapse jumping” phages that passed through olfactory receptor neurons and entered brain tissues, as described in the examples, have had their polypeptides sequenced.

The in vivo screening and selection methods used with sciatic nerves and p75 receptors have been extended, by the inventors herein, to prove that similar methods also function effectively when phage libraries containing random polypeptide sequences are administered to olfactory neurons, which have tips that are accessible in the nasal sinuses, in liquid sprays. Furthermore, subsequent tests showed that at least some phages in the screened libraries were not merely transported into the olfactory neurons, they were also transferred into other neurons, including neurons deep within the olfactory bulb, in the forebrain, and even in the hippocampus.

Accordingly, polypeptide segments containing those types of screened, isolated, and identified amino acid sequences can function as locomotives that can pull and transport molecular complexes into neurons that are fully enclosed within, and protected by, the blood-brain barrier.

For diagnostic purposes in humans, selected types of radiolabeled compounds (such as the ¹²³I isotope of iodine, as one example) can be coupled to the types of polypeptide “locomotives” that will: (1) enter the accessible tips of olfactory-neurons; (2) be transported within the olfactory neurons, at first retrogradely (toward the main cell body), and then by anterograde transport (away from the main cell body), until they reach to the “innermost” synapses of the olfactory neurons; and then, (3) be transferred, via a synaptic transfer, to a different type of cholinergic neuron in the olfactory bulb (in a rat) or in the basal forebrain (in a human). By attaching suitable radiolabeled compounds to those types of polypeptide “locomotives” that can drive endocytosis, neuronal transport, and synaptic transfer, imaging techniques such as “SPECT” scanning can allow diagnostic images to be made, which will indicate whether those types and rates of synaptic transfers are normal and healthy, or abnormally low to a point that indicates a neurodegenerative disease, such as Alzheimer's disease.

Accordingly, this invention involves components or subassemblies that can be summarized as follows:

(1) endocytotic receptor types that are expressed by populations of “targeted” neurons that straddle the BBB and are accessible to liquids that are injected or otherwise administered, such as by nasal spray, etc;

(2) phage libraries, which can be screened using in vivo methods as disclosed herein, to isolate and identify “locomotive” polypeptide segments that will trigger and drive endocytosis, neuronal transport, and synaptic transfer;

(3) suitable imaging agents, which can be coupled to phage particles or to “locomotive” polypeptides that will activate and drive neuronal uptake, axonal transport, and synaptic transfers;

(4) appropriate methods for delivering such imaging agents to specific populations of targeted accessible neurons; and,

(5) appropriate imaging devices, for detecting the locations of (and measuring the quantities and concentrations of) the imaging agents that have been transported, with the help of “locomotive” polypeptides, to one or more classes of “secondary” neurons that are fully inside the BBB.

Each of those components, and their roles in carrying out the invention herein, will be recognized and understood by those skilled in the art, upon reading this disclosure and considering the examples below, and the results as shown in the figures.

### EXAMPLES

### Example 1

### Use of Sciatic and Nasal In Vivo Selection to Isolate Phages with scFv Peptide Sequences that Enable Synapse-Jumping

The in vivo phage screening and selection procedure describes the identification and isolation of phages that contain, in their coat proteins, polypeptide sequences that can trigger and drive all three of the following processes: (i) endocytotic uptake into olfactory receptor neurons; (ii) axonal transport with such neurons; and (iii) synaptic transfer (or “jumping”) out of the directly-accessible olfactory receptor neurons, which straddle the blood-brain barrier (BBB), and other neurons that are not directly accessible to foreign proteins, since they are enclosed within and protected by the BBB.

The nasal screening process began with a phage display library available from Cambridge Antibody Technology. As described in PCT application WO 2003/091387, this library contains huge numbers of differing human gene sequences, from numerous populations reflecting a wide variety of races and ancestries, with protein sequences derived from the “single-chain variable fraction” (scFv) portions of human antibodies. If desired, various other known libraries can be used, to provide the candidate phages. The phages are “phagemid” particles, containing single-stranded DNA that enables them to perform as either a viral-type phage, or as a bacterial plasmid, depending on what types of host cells they are being cultured in. These phages carry an ampicillin resistance gene, and a gene encoding a recombinant human IgG scFv polypeptide, inserted into coat protein III (typically displaying 1 copy per phage particle).

To begin the screening process, a laboratory mouse or rat was briefly anaesthetised, and a phage display library (or portion thereof) was administered into its nasal cavity, in a small volume of sterile saline. The animal was allowed to recover for an appropriate period of time. After periods of time that ranged from minutes to days, the animal was euthanased by overdose of anaesthetic. Selected regions of brain tissue were dissected and removed from the animal, and rinsed thoroughly. The phages in these tissues were isolated by preparing a homogenate or suspension of the tissue, which was then mixed with host cells, comprising the TG1 strain of E. coli bacteria. The bacteria that were infected by phage were isolated and recovered by adding ampicillin, an antibiotic that will kill E. coli cells unless they carry an ampicillin resistance gene, which is carried by the phages.

The number of phages obtained from each sample of brain tissue were counted, and the populations of phage-infected E. coli were replicated, and used to prepare an in vivo phage library that had been enriched by a first round of selection.

The enriched library was then screened in a similar manner, in a second round of screening. The nervous system targeting nasal in vivo selected phage can then be reapplied to the animal for additional round(s) of in vivo selection, or used to produce the ligand that, when attached to the phage, facilitated the transport of the phage from nasal cavity to selected regions of the nervous system.

To obtain the phages that were eventually selected for use by the inventors herein, a fully diverse parent phage library was screened, in the first round, by administering it to a pre-injured sciatic nerve, which was stressed by means of a ligature loop tightened around the sciatic nerve bundle in the hip region of the rat, as described in PCT application WO 2003/091387. This stress provoked upregulation of p75 receptors in the “distal” fibers of the sciatic nerve, and a gel containing the phage library was packed around a severed end of the sciatic nerve bundle, near the knee of the rat. After 18 hours, the rat was sacrificed, and a segment of the sciatic nerve bundle immediately adjacent to the ligature loop was harvested. The phages that had been transported to that location in the sciatic nerve bundle were rescued, and reproduced as follows.

TG-1 E. coli cells that were infected by the phage (acting as a plasmid carrying an antibiotic resistance gene) were removed to a sterile jar containing 25 ml 2TY+2% glucose. After achieving log-phase growth, 25 ml of phage-infected cells was added to 175 ml of glucose-free 2TY medium and grown overnight. The next day, the cell culture was transferred to 50 ml Falcon tubes and centrifuged for 15 minutes at 3500 rpm. The supernatant was discarded and cell pellets taken up in 10 ml of glucose-free 2TY.

To enable phage production, a 10 fold MOI (2.5 microliters) of helper phage M13KO7 carrying the kanamycin resistance gene was added and incubated for 60 minutes at 37° C. with gentle shaking (70 rpm). The phage-infected E. coli were added to a 2 litre shaker flask containing 200 ml of glucose free 2TY+800 μl ampicillin stock (50 mg/ml)+400 ul of kanamycin stock (100 mg/ml) and grown overnight at room temperature (300 rpm) to allow secretion of phage into the medium. The next day, E. coli were pelleted by centrifugation (30 minutes at 10,000 rpm). Phage in the supernatant were precipitated by adding 20% by volume of polyethylene glycol/NaCl, standing overnight at 4° C. Precipitated phage were pelleted by centrifugation (30 minutes at 10,000 rpm). Phage pellets were taken up in 30 ml of sterile Tris-buffered saline (TBS) and re-precipitated by adding 10 ml PEG/NaCl, standing overnight at 4° C. Precipitated phage were again pelleted by centrifugation (30 minutes at 10,000 rpm). Phage pellets were taken up in 8 ml of TBS and 2 ml of PEG/NaCl added, and 500 μl volumes dispensed into 20 sterile screw-cap microfuge tubes. Phage were precipitated overnight at 4° C. Precipitated phage were pelleted by centrifugation (15 min at 14,000 rpm). The supernatant was removed and the vials were sealed. The PEG-precipitated phage pellets were stored at 4° C. for, use in nasal in vivo selection studies. Immediately prior to nasal administration, 50 μl of sterile saline was added to a vial, and the phage were dispersed by trituration.

The enriched phage population was nasally administered to anaesthetised mice, by drawing a 2 μl aliquot of the phage library into a fine plastic pipette, normally used to load samples onto SDS PAGE gels. A recipient mouse anaesthetised using Halothane in air. Immediately after loss of consciousness, the mouse was removed from the halothane, the tip of the pipette was inserted 1 to 2 mm into one nostril, and the liquid contents were rapidly ejected out of the pipette, into the nasal cavity. This operation was quickly repeated with the other nostril, before the animal recovered from the anaesthetic. The animal's tail was marked for identification, and returned to its home cage.

Ten hours later (this delay period was chosen after evaluating the time course for phage transport in mice that were treated in earlier rounds), the mouse was sacrificed by an overdose of halothane vapour, followed by trans-cardiac perfusion with 20 to 25 ml of cold sterile Dulbecco's Medium Eagle's Modification or Dulbecco's phosphate buffer (pH 7.4), using a 23 gauge butterfly needle and a 50 ml syringe. The brain tissues that were freed of blood were dissected into preweighed vials containing known volume of cold tissue lysis buffer (10 mM Tris pH 8.0, 0.1 mM EDTA, 0.1% Triton X-100). Preferably, tissues were homogenised by trituration through the pipette tip, and used to infect TG-1 E. coli cells. Alternatively, some tissue samples were frozen at −70° C. until the researchers were ready to isolate the phage in the tissue samples; frozen tissue samples were removed from the freezer and, without thawing, embrittled and ground to fine powder, in a mortar and pestle chilled with liquid nitrogen, before the tissue could thaw, and the phages were then used to infect host TG-1 E. coli.

After trituration to homogeneity in phage lysis buffer, dilutions of tissue homogenate samples were prepared without first pelleting the insoluble cell debris, because significant numbers of phage were consistently recovered when the E. coli cells were added to cell debris pellets that had been washed and resuspended. To avoid reducing the viability of E. coli, the volume of tissue homogenate was no more than 20% of the volume of the host cell culture (freshly prepared TG-1 E. coli in 2TY+2% glucose in log growth phase, optical density approximately 0.2 at 600 nm). Capped centrifuge tubes were gently mixed (30 rpm) at 37° C. for 60 minutes in a shaking incubator, to allow the phage to infect E. coli. Ampicillin stock (50 mg/ml) was then added to final concentration of 100 ug/ml and phage infected E. coli culture spread evenly on petrie dishes or 234 mm×234 mm Nunc tissue culture plates containing 1.5% agar, 2TY medium, 2% glucose and 100 ug/ml ampicillin. At least two tissue samples at three dilutions were prepared together. Culture plates were sealed with parafilm and incubated overnight at 30° C. in the shaking incubator.

Within 18 hours, colonies were 0.5 to 2 mm in diameter, without evidence of secondary colonies. Plates were removed and chilled to 4° C., and the number of colonies per plate was counted within 24 hours. The number of phage in each tissue sample was determined from at least two sequential titering experiments. Phage titre counts were used to calculate the mean number of phage recoverable per animal from discrete tissue regions.

After counting, for each tissue sample, 96 randomly selected colonies were individually picked into wells of a sterile deep well plate containing 96 wells, each well containing 0.5 ml of 2TY, 2% glucose, and 100 μg/ml ampicillin. Clones were grown overnight at 37° C. with gentle shaking. The next morning, 500 μl of sterile 50% glycerol was added and 200 μl from each well was removed to a second 96-well plate, to prepare a duplicate 96-well plate. The two plates were frozen at −80° C. until ready for sequencing.

After picking colonies for sequence analysis, a sterile bent glass pipette was used to scrap all colonies of the plate into 5 ml of 2TY in a shallow dish. Five ml of glycerol was added, then 10×1 ml aliquots of phage-infected E. coli were prepared, and frozen at −80° C. If another round of in vivo phage selection was planned, one aliquot of phage-infected E. coli was used to prepare the next round of phage.

To examine the tissue distribution of selected phage, the fluorescent tracer molecule known as FITC (purchased from Sigma Chemical) was coupled to a phage population. Briefly, PEG-precipitated phage were dissolved in sterile saline and applied to a 10 ml Sephadex G-25 column that had been pre-equilibrated with Dulbecco's PBS, pH 7.4. Phage were collected in 1.6 ml aliquots. The amount of phage present, after desalting, was estimated from absorbance at 280 nm, assuming an extinction coefficient of 1 mg/ml 1.25 and diluted to 1 mg/ml. 1.0 mg of FITC, dissolved in 100 μl dimethyl sulfoxide, was added to 1 mg of phage. The reaction vial was wrapped in aluminium foil, and the binding reaction proceeded for 1 hour at room temperature with gentle mixing. Phage were then precipitated overnight at 4° C. after adding 20% volume of PEG/NaCl. Precipitated phage were recovered by centrifugation (14,000 rpm at 15 min), taken up in 800 μl of Tris-buffered saline, and again precipitated overnight at 4° C. following addition of 200 μl of PEG/NaCl. The PEG precipitation step was repeated (usually 2 or 3 times) until no significant FITC could be seen in the supernatant.

FITC-labelled phage were nasally administered to mice using the same procedures described above, except that after trans-cardiac perfusion with cold Dulbecco's phosphate buffer (pH 7.4), animals were perfusion-fixed with 50 ml of ice cold 0.1M sodium phosphate buffer pH 7.4 containing freshly-mixed 2% paraformaldehyde and 0.2% parabenzoquinone (Conner 2002). Tissues of interest were then exposed by partial dissection, then immersion-fixed on ice in the same fixative for 2 hours.

Complete dissection followed, with tissues being cryoprotected in 30% sucrose in Dulbecco's PBS before embedding in a cutting compound and frozen-sectioned, using a cryostat.

Some tissues (such as nasal epithelium) were decalcified by immersion in 4% Na₂-EDTA (pH 7.2) for 2 to 6 weeks, before cryoprotection and histological processing.

Tissues sections were thaw-mounted onto 4% gelatin coated slides and examined using a fluorescent microscope.

One set of tests indicated that large numbers of phages (well over 10,000, based on titering tests) appeared very rapidly (within about 2 minutes) in olfactory bulb tissues. These same tests also indicated that the phages were cleared rapidly from the olfactory bulb tissues; concentrations dropped by about 80 to 85% of their peak observed values within 30 minutes, and by more than 90% within 60 minutes. Observations showed that, as the phages were cleared from the olfactory bulb tissues, they were dispersed to various other tissues, mainly in adjacent or nearby regions of the brain, and appeared in those other brain regions at lower concentrations.

Accordingly, since olfactory bulb tissues held the highest concentrations, but only briefly, such tissues from several mice were pooled, and used to prepare a derivative scFv display library that was enriched for scFv phage that had indeed reached and entered olfactory bulb tissues (thereby demonstrating endocytotic uptake by, and axonal transport through, the olfactory receptor neurons). This pooled library was used in subsequent studies that aimed to determine whether the selected phages could undergo transneuronal transport, into neurons that lie wholly within the blood brain barrier.

One such library was designated as the 041207-OB (10 hr)/sciatic (18 hr)/diverse library, to indicate that it arose from a complete library that went through sciatic nerve screening with an 18 hour delay, followed by nasal screening with a 10 hour delay, and it was deemed to be complete and ready for subsequent usage and testing on Dec. 7, 2004.

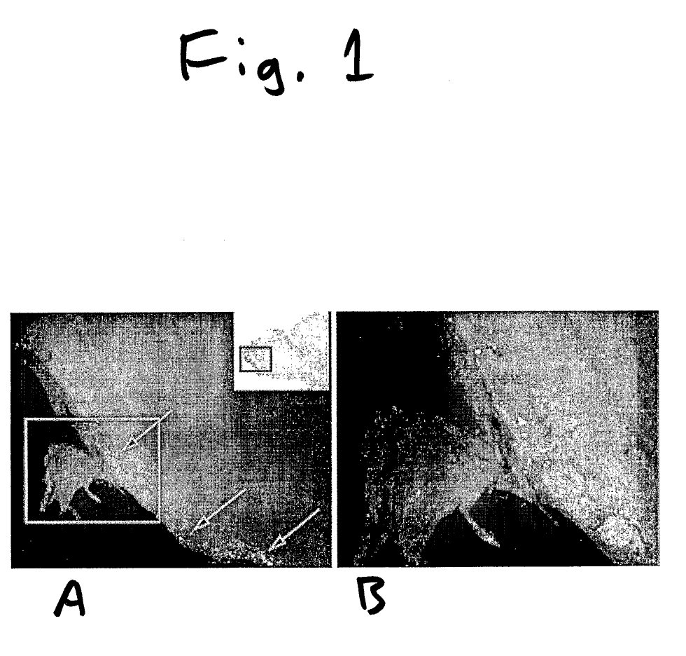

Accordingly, FIG. 1 is a fluorescent photomicrograph showing olfactory tissue, 5 minutes after nasal administration of fluorescent FITC-labelled phages. FIG. 1A contains an inset showing the general location of FIG. 1A near the lower front region of the olfactory bulb. FIG. 1B is an enlargement of the boxed region shown in FIG. 1A.

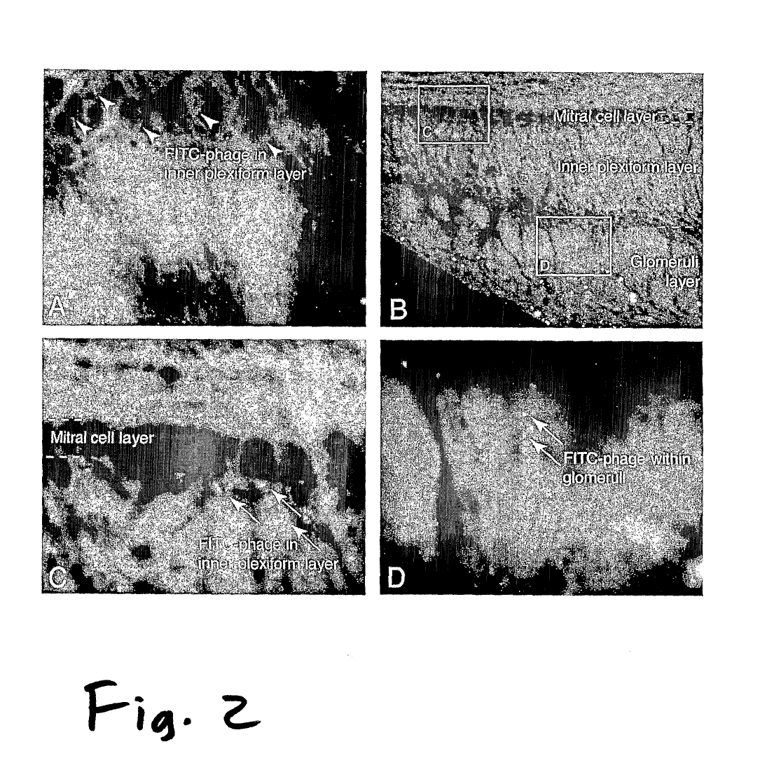

FIG. 2 also is a fluorescent photomicrograph, shown FITC-labelled phages that remained in various portions of the olfactory bulb after 30 hours. FIG. 2A shows phages that have penetrated beyond a superficial layer known as the olfactory glomeruli, into a layer known as the inner plexiform layer, which is labelled in FIG. 2B. FIG. 2B also shows the locations of the enlarged segments shown in FIGS. 2C and 2D. FIG. 2C shows the mitral cell layer, which is deeper still than the inner plexiform layer. FIG. 2D shows an inner portion of the olfactory glomeruli layer.

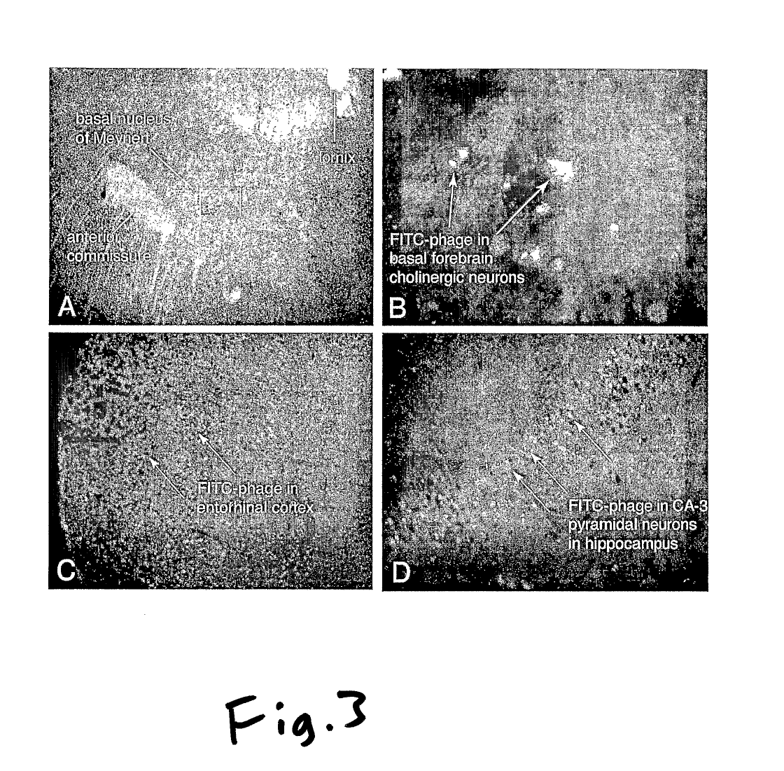

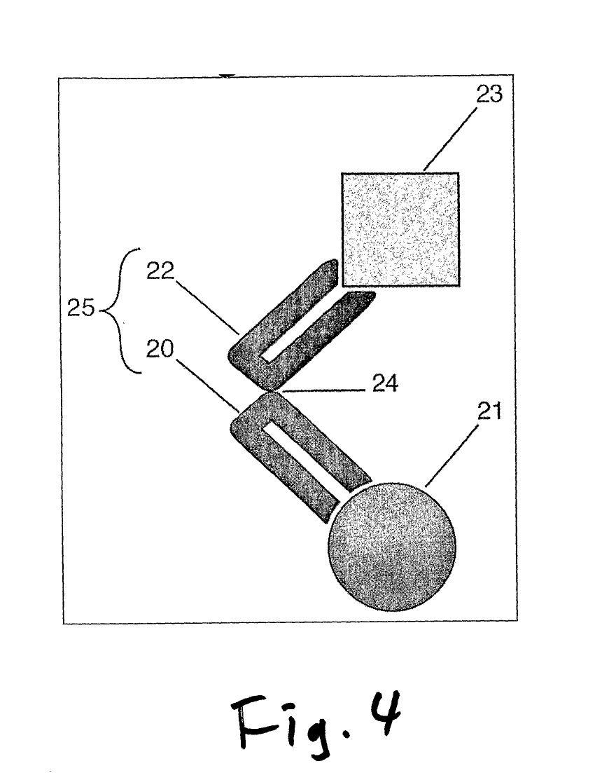

FIG. 3 shows the presence of FITC-labelled fluorescent phages that are still deeper in the brain tissue, harvested at 30 hours after nasal administration. FIG. 3A shows the hindlimb of the diagonal band of Broca, and the basal nucleus of Meynert. FIG. 3B is an enlargement of the region shown by the rectangle in FIG. 3A, and clearly shows labelled phage that have reached cholinergic neurons in the basal forebrain of the mice. FIG. 3C depicts phages that have reached the entorhinal cortex, and FIG. 4D depicts phages that have reached the CA3 layer of the hippocampus.

Accordingly, these phages have fully demonstrated “synapse-jumping” capabilities, and can be used both for diagnosis and monitoring (if properly labelled for SPECT or similar imaging), and also for delivery of therapeutic polypeptides and other drugs into portions of the central nervous system that are fully protected by the blood-brain barrier.

In accord with the disclosures and findings summarized above, regarding the selection and creation of a phage library that clearly has demonstrated uptake, transport, and synapse-jumping capability, FIG. 4 depicts a molecular complex 25 that can be used to deliver diagnostic, therapeutic, or other drugs into BBB-protected brain or spinal tissue. Molecular complex 25 comprises: (i) a “locomotive polypeptide” 20, which has demonstrated uptake, transport, and synapse-jumping capability, and which can bring to a surface protein 21 on a BBB-straddling cell, such as an olfactory receptor neuron, and (ii) a second polypeptide 22, which can reversibly bind to a drug molecule 23. The two polypeptide segments 20 and 22 can be coupled to each other via a covalent bond or linker molecule 24. Alternately, a single-chain polypeptide can be created that has a first domain that will function as the “locomotive polypeptide” 20, and a second domain that will function as the “drug carrier” polypeptide 22.

### Example 2

### Use of P75 Receptor Ligands for Diagnosis or Monitoring of Disorders Involving Lower Motor Neurons

At present, diagnosis and monitoring of ALS relies on use of physical examination, and there exists no method for selectively labelling and imaging motor neurons that become stressed or dying, in ALS patients (or in animal models of ALS that use in vivo imaging, rather than animal sacrifice followed by tissue analysis). Availability of a method for selectively labelling and imaging ALS diseased motor neurons may be expected to provide additional information that will assist the clinician in diagnosing the neurological condition and monitoring its progress.

A well-known neuronal receptor designated as p75 is known to be “upregulated” in lower motor neurons that are suffering from stress, as occurs in patients suffering from amyotrophic lateral sclerosis (ALS), or from traumatic peripheral motor nerve injuries (Seeburger et al 1993; also see WO 2003/091387). Thus, the p75 system can be used as a marker of ALS-diseased neurons.

Because of the known importance of the p75 receptor system, various researchers have created monoclonal antibodies that bind to it. One such monoclonal antibody, known as 192-IgG (described in Chandler et al 1984), has been shown to bind with high affinity to p75 receptors in rats. That monoclonal antibody, and various smaller fragments derived from it, have been shown to undergo endocytosis and retrograde axonal transport, in peripherally-projecting neurons that express the p75 receptor (Yan et al 1988).

In this method, a radiolabelled p75 receptor ligand that stimulates p75 receptor endocytosis and retrograde transport (such as a p75-binding monoclonal antibody or fragment as described above, or a polypeptide segment isolated from a phage library by the in vivo screening methods described in WO 2003/091387) is administered to accessible terminals of lower motor neurons, by means such as injection into muscle tissue, or by intravenous injection into circulating blood, if suitable dosages are used. Where the lower motor neuron expresses abnormally high levels of p75, the radiolabelled p75 receptor ligand will stimulate internalisation and retrograde axonal transport within the neurons, allowing the neuronal cell bodies to be visualised, within the spinal cord, by making use of an imaging system such as single photon computerised tomography (SPECT). The amount and location of radiolabelled p75 receptor ligand accumulating in the spinal cord allows identification of the number and location of lower motor neurons that are expressing abnormally high levels of p75. As expression of p75 is upregulated following peripheral nerve injury or in certain disease states such as in ALS, this method enables diagnosis or monitoring of these neurological disorders.

Animal-model time course studies predict that significant retrograde concentration of a radiolabeled p75 ligands in the spinal cords of humans will take place about 12-24 hours after bolus administration. Repeated SPECT imaging may be undertaken at a range of times to assist the clinician in monitoring the axonal transport process as well as the location and number of p75 expressing motor neurons in the spinal cord.

A radioisotope with a half-life that will be compatible with the time course of p75 ligand travel is ¹²³I, an isotope of iodine, which reportedly has a half-life of 13 hours and 159 KeV gamma rays (e.g., Kung 1991), and which is well-suited for SPECT imaging. Two of the most commonly used brain perfusion agents used for SPECT imaging (known as ¹²³I-IMP and ¹²³I-HIPDM) pass readily through the BBB. Various methods are known for attaching ¹²³I and similar labels to the aromatic ring of tyrosine residues, in p75 ligand polypeptides; one such method, the lactoperoxidase method, is described in Ferguson et al 1991.

### Example 3

### Use of P75 Receptor Ligands for Diagnosis or Monitoring of Disorders Involving Basal Forebrain Cholinergic Neurons

Brain imaging procedures capable of diagnosing and/or monitoring Alzheimer's disease are progressing rapidly, but none have reached a point of being optimal and ideal, as discussed in articles such as Knopman et al 2001, DeKosky and Marek 2003, Klunk et al 2003, and Mathis et al 2005. Accordingly, the methods and agents disclosed herein offer another set of tools that can be evaluated and used, in coordination with other methods and agents that have been or are being developed.

In addition to being involved in ALS and peripheral traumatic peripheral motor nerve injuries (as described in Example 1) the p75 receptor system is also known to be involved in cognitive impairment and Alzheimer's disease (e.g., Mufson et al 2002). However, the effects seen in the p75 receptor system, in those different classes of disorders, are quite different. In ALS and peripheral motor nerve injuries, p75 receptors are upregulated; by contrast, in Alzheimer's disease and cognitive impairments, p75 receptors are present in abnormally low numbers.

Accordingly, ¹²³I-labelled endocytotic p75 receptor ligands (as described above) can be nasally administered to accessible terminals of olfactory receptor neurons. The labelled ligands will be taken up by olfactory receptor neurons, and will undergo axonal transport into the olfactory bulbs of rats, or into the basal forebrains of humans. Within those BBB-protected brain regions, some of the labelled p75 ligands may be synaptically transferred to other neurons that interact directly with the olfactory receptor neurons, and other labelled p75 ligands may be released into the cerebrospinal fluid, allowing them to bind to p75 receptors expressed on the surfaces of neurons in those regions of the brain. In either case, SPECT imaging will allow the pathways and rates of such transport to be tracked and monitored, at repeated intervals during a span of about 24 hours after administration of the labelled ligands. This will allow researchers and diagnosticians to correlate and compare the patterns and rates of p75 ligand travel that are observed, in particular animals or patients, with similar and differing patterns and rates that have been correlated with other animals or patients suffering from cognitive impairments, Alzheimer's disease in humans, and Alzheimer-modeling syndromes and symptoms in animals, at various known levels of severity. Accordingly, abnormalities in p75 ligand travel patterns and rates, following nasal administration, will allow clinicians and others to diagnose and monitor neurological disorders involving basal forebrain cholinergic neurons, in humans.

### Example 4

### Use of P75 Receptor Ligands to Diagnose or Monitor Neurological Disorders Involving Upper Motor Neurons

The following imaging procedure describes how to use p75 receptor ligands to diagnose or monitor a neurological disorder in which axonal transport in upper motor neurons is abnormal. An example of such a neurological disorder is paralysis due to stroke or traumatic head injury.

In this method, a radiolabelled of a polypeptide called neurotrophin-3 (abbreviated as NT-3) is complexed with a drug delivery carrier having a bi-specific antibody design. This complex is administered to accessible terminals of lower motor neurons. The bi-specific antibody drug delivery carrier is used to deliver the radiolabelled NT-3 into the spinal cord. Released within the spinal cord, the radiolabelled NT-3 can undergo receptor mediated internalisation and retrograde transport within upper motor neuron processes terminating on the lower motor neuron. In normal healthy upper motor neurons, the radiolabelled NT-3 will undergo retrograde axonal transport to the upper motor neuron cell bodies in the motor cortex. The process of uptake and retrograde axonal transport of the radiolabelled NT-3 can be visualised within the spinal cord by making use of an appropriate medical imaging device such as single photon computerised tomography (SPECT). In certain upper motor neuron disorders in which axonal transport is abnormal, the uptake and retrograde axonal transport of radiolabelled NT-3 will be abnormal. The abnormal uptake and retrograde axonal transport of the radiolabelled NT-3 can be detected by the medical imaging device and so assist the clinician in diagnosing the upper motor neuron disorder and in monitoring the health of the upper motor neurons in human subject.

### Example 5

### Sequence Analysis of Phage Display Library Used for Nasal In Vivo Phage Selection

Example 1 described how synapse-jumping scFv phage could be isolated, comprising a subpopulation of scFv phage that underwent internalisation and retrograde axonal transport with the rat sciatic nerve as described in WO/2003/091387.

After that selected population had been isolated, analysis of the scFv amino acid sequences in the selected library was carried out. The results indicated that: (a) the preferred anti-p75 scFv for use in this invention has an apparent low affinity of binding (putative fast-on, fast-off rate); and, (b) it could not be isolated by conventional in vitro panning methods which rely on selecting for high affinity binding.

In more detail, individual clones from phage populations were isolated following in vivo phage selection, and also following conventional in vitro panning. The amino acid sequences of individual clones were characterised by measuring the strength of their binding to p75NTR, in vitro, by ELISA assays. The selection pressures at work in in vivo phage selection method and conventional in vitro panning were deduced from changes in the relative proportion of individual clones in random samples taken from the phage populations.

ELISA assays were carried out as follows. Conventional in vitro panning and sciatic in vivo selection methods, as described in WO/2003/091387 and elsewhere in this specification, were performed. Individual colonies were picked into 100 μl of 2TYAG in a 96 well plate and grown at 30° C. shaking at 100 rpm overnight. The cultures in this plate were used to inoculate a fresh deep well block containing 900 μl of 2TYAG and 50 μl of 50% v/v glycerol was added to the original plate and it was stored frozen at −70° C. The fresh plate was grown at 37° C. for 5-6 hours until the cultures were turbid. To each well of the replica plate 100 μl of M13KO7 in 2TYAG (at 5×10¹⁰ Pfu/ml, an m.o.i. of 10) was added. The plate was incubated for 30 min without shaking at 37° C. and then for 30 min with shaking (100 rpm) at 37° C. to allow superinfection of the helper phage. The cultures were pelleted at 2000 rpm for 10 min and the supernatant discarded. The bacterial pellets were resuspended in 1 ml of 2TYAK and grown overnight at 30° C. shaking at 280 rpm. The phage was blocked by the addition of 200 μl of PBS/18% Marvel, incubated for 1 hr at room temperature. The plates were spun at 3200 rpm for 10 minutes and the phage containing was supernatant used directly. Antigen plates were prepared by adding 50 μl of 1 μg/ml recombinant human p75NTR/FC chimera (RnD systems) in PBS to microtitre plate wells and incubating overnight at 4° C. Plates were washed 3× in PBS and blocked for 1 hr in 3% MPBS at RT. 50 μl of preblocked phage were added to each well. The plates were incubated stationary at room temperature for 1 hr after which the phage solutions were poured off. The plates were washed 3 times in PBS using a plate washer. To the ELISA plate well, 50 μl of a 1 in 5000 dilution of the anti-M13-HRP conjugate (Pharmacia) in 3 MPBS was added and the plates incubated at room temperature stationary for 1 hr. Each plate was washed as described. 50 μl of TMB substrate was then added to each well, and incubated at room temperature until a distinct colour change was observed in some wells, after which the reaction was stopped by the addition of 50 μl of 1M H₂SO₄. The absorbance signal generated by each clone was assessed by measuring the optical density at 450 nm using a microtitre plate reader. Positive clones were detected by comparing the ELISA signal to that of a control well coated with 1 μg/ml recombinant TRAIL R2 receptor/FC chimera (RnD systems). ELISA scores were calculated by dividing the reading of p75NTR by TRAIL R2 receptor.

DNA sequencing of scFv antibodies was carried out as follows. The nucleotide sequences of the scFv antibodies were determined by first using vector-specific primers to amplify the inserted DNA from each clone. Cells from an individual colony on a 2TYAG agar plate were used as the template for a polymerase chain reaction (PCR) amplification of the inserted DNA using the primers pUC19reverse and fdtetseq. Amplification conditions consisted of 30 cycles of 94° C. for 15 sec, 55° C. for 1 min and 72° C. for 45 sec, followed by 7 min at 72° C. The PCR products were cleaned up using shrimp alkaline phosphatase and Exonuclease I. The sequencing reaction used Big Dye Terminator V3.1 (Applied Biosystems) and the primer gene3leader. The sequence was read using a 3700 DNA Analyser (ABI). Sequences were analysed using Blaze2.

The results are provided in Tables 1-3.

It was hypothesized that antibodies with higher ligand binding affinity in vitro would be the most efficient stimulators of internalization in vivo. To test that hypothesis, an affinity rank score for individual clones was first generated using ELISA score data: scFv with a higher affinity rank score of one indicates strongest binding to p75NTR in vitro. The ELISA score was used to test for a correlation with the expansion score (Table 1). No correlation was observed between ELISA score and expansion score (Spearman rank correlation coefficient, r=−0.098; Student two tailed t test: t=0.01; less than 1% level of significance; FIG. 3a). A similar lack of correlation (r=0.089) was observed when the internalization rank score data was used. This indicates that the strength of scFv binding to p75NTR did not predict the propensity to stimulate internalization.

Listed in Table 1 are 18 unique internalising anti-p75NTR antibodies isolated from one round of in vivo selection. Internalisation rank v.s. ELISA rank: Rank correlation coefficient r=−0.15. Student/Es two-tailed t-test t=0.01 indicated less than 1% level of significance. Expansion rank v.s. ELISA rank: r=0.089, t=0.01. Taken together, these data indicated that there was no linear relationship between ELISA scores and internalisation efficiency.

Rank correlation coefficient was calculated using the formula r=1−{(6 sigma d2)/(n(n2−1))} where d=difference between the ranks of paired observations, n=number of paired observations. 1 sigma r−1 where r=1 indicated a perfect correlation, whereas r=−1 indicated a perfect inverse correlation. r=0 indicated no correlation. Student's t-test was calculated using the formula t=r/{(1−r2)(n−2)}½ (e.g., Hamburg 1987, Statical Analysis for Decision Making, HJB Publishers).

Each round of in vitro panning increased the proportion of antibodies with ELISA scores above 1.5. Given that 95% of the internalizing anti-p75NTR have an ELISA score of less than 1.5, in vitro panning progressively decreased the proportion of internalizing antibodies in the population and the complete elimination of frequently occurring internalizing antibodies by the third in vitro panning round.

Table 2 contains a list of frequently occurring antibody clones that was isolated from three rounds of in vitro panning against recombinant human p75NTR. Only some antibodies were shown to be internalised through in vivo selection.

Finally, it was predicted that anti-p75scFv from pre-injured nerve could be isolated following application of the parent, fully diverse (not in vitro panned) library. A number of frequently occurring clones were isolated with identical sequences to those isolated following in vitro panning against p75NTR (Table 3). All these anti-p75NTR exhibited an ELISA score below 1.5. In addition to the anti-p75NTR, a large number of other clones were isolated with sequences that did not correspond to known anti-p75NTR.

Unexpectedly, it was found that low affinity antibodies were more efficient stimulators of internalization than high affinity binders. None of the low affinity antibodies would have been identified, if only conventional in vitro panning were used. Indeed, none of the high affinity antibodies generated by the standard procedure of repeated rounds of in vitro panning exhibited an ability to stimulate internalisation.

The identified class of internalisation stimulation antibodies are characterised by exhibiting a binding strength of low affinity as determined using in vitro ELISA assays but an ability to stimulate internalisation, and hence delivery into the target cell, of a payload of attached other proteins and DNA (in this case a bacteriophage). The high affinity antibodies are characterised by exhibiting a binding strength of high affinity but no ability to stimulate internalisation.

The in vitro panning procedure is directed towards isolation of clones with high affinity binding based on the assumption that antibodies need to exhibit high affinity binding to be useful for therapeutic purposes. This assumption is challenged by the finding that anti-p75 scFv with low, not high, affinity are useful for delivering payloads (bacteriophage) into neurons in vivo.

The function of antibodies depends on their specificity and affinity for antigen (Foote and Eisen 2000) and the importance of affinity maturation to develop high affinity antibodies is well recognized (eg Ellmark et al., 2000). Conventional in vitro panning can isolate high affinity antibodies from phage libraries but cannot be used to isolate the low affinity antibodies for stimulating p75NTR internalization.

It is widely assumed that high affinity and high specificity are equivalent and that antibodies must exhibit both a high rate of attachment and a slow dissociation rate to be useful. Indeed, the in vitro panning methods select for such binding characteristics because antibodies with a high rate of dissociation are lost during repeated rounds of washing. In contrast, antibodies with both a high rate of association and a high rate of dissociation can be isolated by the in vivo phage selection method where those receptor-binding characteristics are sufficient to stimulate internalization. For example, NGF, the endogenous ligand for the p75NTR, which normally stimulates internalization and retrograde axonal transport (Johnson et al., 1987), exhibits both a fast association and a fast dissociation rate constant (Eveleth and Bradshaw, 1988); indeed it was previously called the fast NGF receptor to distinguish it from the higher affinity slow receptor (later discovered to be the trkA receptor (Kaplan et al., 1991).

The preceding discussion focuses on the low affinity neurotrophin receptor, p75NTR. A cursory analysis of the polypeptide sequences recovered from within the nerve after applying the fully diverse parent phage display library (ie not enriched for molecules to any specific antigen such as p75NTR), demonstrated the presence of polypeptide sequences that stimulate internalization but do not recognize p75NTR. Therefore, cell surface molecules other than p75NTR that will undergo internalization exist and the methods disclosed herein can be used to identify and characterize the internalization stimulating polypeptides attaching to cell surface molecules of initially unknown identity.

### Example 6

### Use of Nasal In Vivo Phage Selection to Isolate NALT Targeting Phage as Immunological Adjuvants for Nasal Vaccination

The nasal mucosa is the first site of contact with inhaled antigens; accordingly, “nasal-associated lymphoid tissue” (abbreviated as NALT) can play a major role in the stimulation of both local (IgA) and systemic (IgG) immune responses, as reviewed in Kuper et al 1992. In humans, nasal administration of antigen stimulates a stronger IgG production response (and with less antigen) than other mucosal immunization routes (Kozlowski et al 2002). Accordingly, administration of vaccines via nasal sprays offers a promising way to benefit public health. Among other advantages, it can eliminate the use of needles, and the problems that accompany needles (including sterilization, disposal, lack of appeal among people who should be immunized, theft and abuse by drug addicts, etc.).

However, intranasal immunization has not lived up to its potential, because of various limitations and shortcomings. Among other factors, adverse reactions involving inflammation of the olfactory bulbs after intranasal administration have been observed in some cases (Van Ginkel et al 2000), and better intranasal adjuvants are needed (Lang 2001; Levine 2003).

In this field of medicine, adjuvants are substances that, when used in combination with an antigen, produced a stronger and more robust immune response than can be produced by the antigen alone. In general, their role is to signal the immune and/or inflammatory system that something has created a problem and needs attention, in a certain specific region of tissue; then, when the immune and/or inflammatory system responds to the provocation, the responding cells will encounter the antigen, and will begin mounting an immune response that attacks the antigen. Adjuvants are well-known, and are part of standard practices whenever antibodies are being created in laboratory settings. They can be divided into two general classes, based on their mechanism of action: (i) vaccine delivery adjuvants increase immune responses by increasing exposure levels between antigens and antigen-presenting cells (APC); and, (ii) immunostimulatory adjuvants activate the immune system by stimulating the release of cytokines, stimulating the expression of co-stimulatory molecules, or provoking similar effects (O'Hagan et al 2001).

The concept of using bacteriophage as a platform for adjuvant development is not contemplated by experts in vaccine development in industry. For example, in a recent review of mucosal adjuvants and delivery systems (Vajdy et al 2004), there was no mention of the potential use of bacteriophages, as adjuvants. That review notes that microparticulate formulations with adsorbed antigens are particularly potent means of achieving antigen delivery into APC; it reviews the literature describing available microparticle delivery systems, and it notes that the size, surface charge, and hydrophobicity of microparticles affects their phagocytosis by macrophages. However, it does not suggest that phages could be used to provide comparable types of microparticles, or how phages might be manipulated to render them even more effective, when used in such a manner.

Despite that lack of interest in the prior art, it is disclosed herein that phage display librarys can be processed, by in vivo screening and selection methods, to isolate and identify certain phages that will be efficiently internalised, and that can be used to provide immunostimulatory adjuvants.

If an intranasally-administered antigen is soluble, it may be able to penetrate the nasal epithelium and interact with APC (such as macrophages or dendritic cells), which can then migrate to superior or posterior cervical lymph nodes, present antigen to T cells, and initiate the immune response cascade. If the antigen is of a particulate nature, it may adhere to specialised epithelial cells, the “microfold” (M) cells that are concentrated in the epithelium above the NALT tissue. The thick glycocalx barrier overlying much of the epithelium is absent over M cells, and M cells serve as a portal for antigen passage through the mucosa, as reviewed in Man et al 2004. The M cells internalise and transport the particulate antigen to the underlying NALT. The NALT contains T- and B-cells as well as APC that drain to the lymph nodes and stimulate immune response cascade. Because M cells efficiently transport antigen across the epithelial cell barrier, M cell targeting ligands may be used to increase the efficiency of antigen delivery to APC.

The following nasal in vivo phage selection procedure describes how to isolate phage displaying peptides that target delivery of attached passenger molecules to the immune system. This method was used with a Fd88-15mer library (Smith 1988). The Fd88-15mer library used phage particles containing ssDNA with an tetracycline resistance gene and gene encoding random 15 amino acid peptide, inserted into the gene encoding for coat protein VIII (typically displaying peptide 25-40% of the coat protein VIII on each phage particle).Other phage display libraries may substituted with phage titering, expansion, and other methods appropriate to the substitute phage library.

To use this 15-mer library, a laboratory mouse is briefly anaesthetised and a phage display library or derivative is administered into its nasal cavity, as described above in Example 1. The animal is then allowed to recover for an appropriate period of time (in a range of minutes to days, depending on the tissues of interest) before the animal is sacrificed. Selected regions of the immune system (such as lymph nodes, etc.) are dissected from the animal, and phages in these tissues are isolated by preparing a homogenate or suspension of the tissue and mixed with host K91 E. coli. E. coli infected by phage are recovered by adding the antibiotic tetracycline, which will kill all E. coli except those that have been infected by the phage, which carry an antibiotic resistance gene. The number of phage in the tissue can then be counted and the population of phage-infected E. coli expanded and used to prepare an in vivo selected phage library. The nasal in vivo selected phage can then be reapplied to the animal for one or more additional round(s) of in vivo selection, or it can be used to produce a ligand that, when attached to the phage, will facilitate the transport of the phage from the nasal cavity, to immune tissue.

To carry out this project, the methods described in Example 1 were used, with the following differences. Using standard procedures, a master and working cell bank of E. coli K91 (G Smith) was prepared and frozen at −70° C. This was then used to prepare master and working cell banks containing the phage library. A 200 μl aliquot of the phage library to 30 ml culture of E. coli K91 grown overnight in a shaking (300 rpm) 37° C. incubator to stationary phase, in LB medium. One hour prior to addition of phage, the culture was allowed to rest, to allow E. coli to regrow their sheared pilli. Infection was allowed to proceed for 1 hour before adding the phage infected E. coli to 220 ml of chemically defined medium containing 12.5 ug/ml tetracycline in a shaker flask. The culture was allowed to grow overnight to stationary phase with vigorous shaking (300 rpm) to shear pilli and reduce probability of superinfection of E. coli by phage particles in the medium. The culture was then chilled on ice and an equal volume of glycerol containing chemically defined cell freezing mixture added. Twenty-five ml aliquots of the E. coli containing phage were frozen in liquid nitrogen and defined as the master cell bank (MCB).

A working cell bank (WCB) was prepared by thawing an aliquot of the MCB and adding it to 225 ml of chemically defined medium containing 12.5 μg/ml tetracycline for overnight culture with vigorous shaking. The culture was chilled, a chemically defined cell freezing solution added, and aliquots prepared as for the MCB. This was defined as the WCB.

The phage library for in vivo use was prepared using the following procedure. An aliquot of the WCB was thawed and added to a 1 litre bioreactor with 975 ml of chemically defined medium for high cell density cultures using sorbitol as a carbon source plus 12.5/ug/ml tetracycline. Culture was grown for 48 hours to stationary phase at room temperature with vigorous aeration and high shear on a magnetic stirrer to reduce probability of superinfection of E. coli by secreted phage. The culture medium was then chilled on ice and centrifuged at 10,000 rpm for 30 min to pellet E. coli. Twenty percent by volume of PEG/NaCl (Smith 1988) was added to supernatant and chilled overnight to precipitate the phage. PEG precipitated phage were pelleted by centrifugation at 10,000 rpm for 30 min and the supernatant discarded. Further PEG precipitation of phage was essentially as described under Example 1.

For the nasal in vivo phage selection and titering methods, the methods described under Example 1 were used with the following differences. After homogenisation, phage were rescued by allowing them to infect host K-91 E. coli (freshly prepared K91 E. coli in LB medium+2% glucose in log growth phase, OD approx 0.2 at 600 nm). Capped centrifuge tubes were gently mixed (30 rpm) at 37° C. for 60 minutes in a shaking incubator to allow phage to infect E. coli. Tetracycline stock (20 mg/ml) was then added to final concentration of 12.5/ug/ml and phage infected E. coli culture spread evenly on petrie dishes or 234 mm×234 mm Nunc tissue culture plates containing 1.5% agar, LB medium, 2% glucose and 12.5/ug/ml tetracycline. Phage were plated and titred essentially as for Example 1, except LB medium was used.

For the tracer studies, the methods described under Example 1 were used with the following differences. Following perfusion fixation, tissues dissected out for histological analysis included head tissue (in sagittal section), lung, liver, muscle, skin, and spleen; heads in sagittal section were decalcified by immersion in 4% Na2-EDTA pH 7.2 for 2 to 6 weeks before cryoprotection and histological processing.

A 15-mer peptide library of phages applied to 10 mice, and olfactory bulbs were dissected soon thereafter, mean time 40±10 min (standard deviation). Olfactory bulb tissues were pooled and used to prepare a derivative peptide display library enriched for cellular and tissue binding phage. This library was used to isolate NALT targeting peptide phage

The phage were intranasally administered to 10 mice and olfactory bulb and NALT tissue (Asanuma et al 1997) dissected out at 45±6 min into phage lysis buffer and then used to infect host cells for 1 hour. The putative NALT targeting phage display library was prepared using procedures as described above. Phage were FITC-labelled and intranasally administered, and animals were perfusion-fixed at 30 min or 2 hr, and processed for fluorescence microscopy to confirm the isolation of NALT targeting peptide display phage. To test whether inactivated phage particles can be used as an adjuvant, the NALT targeting phage were heat-inactivated in a boiling water bath for 10 minutes, before FITC conjugation and animal testing.

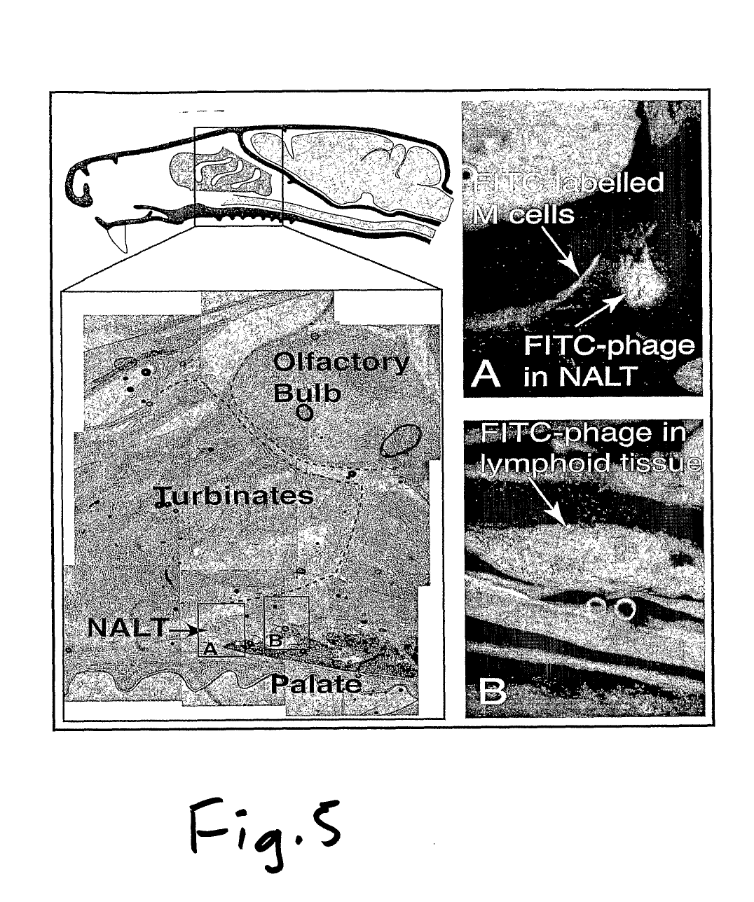

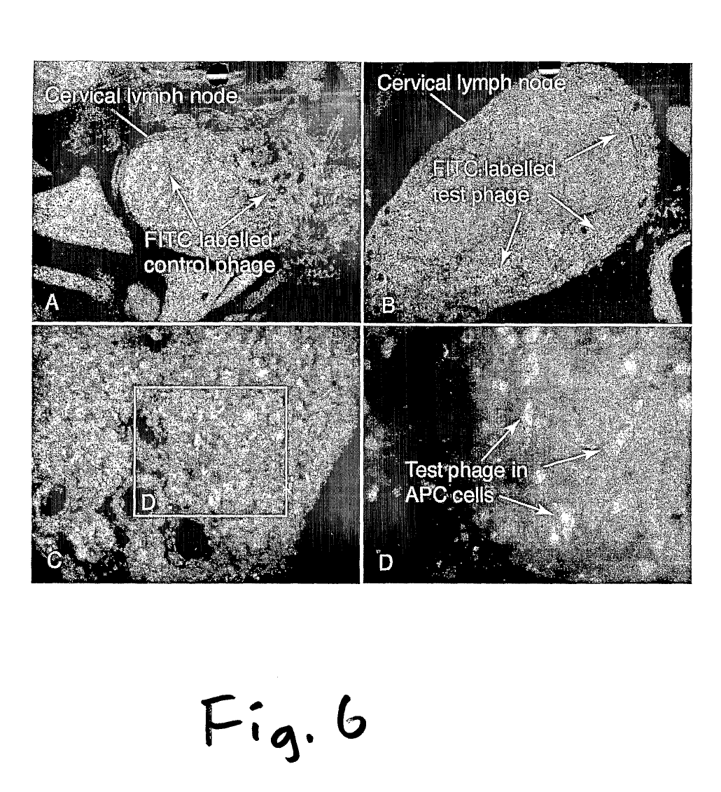

FIGS. 5 and 6 demonstrate that NALT targeting phage were isolated by in vivo selection.

The time course of appearance of FITC-labelled NALT targeting phage, in the cervical lymph nodes, was consistent with these phage entering the NALT within 1 hour of nasal administration, involving uptake by antigen presenting cells, and migration by lymph drainage to the cervical lymph nodes, as part of the normal immune response. Given that heat-inactivation of NALT targeting phage apparently did not adversely affect this process, inactivated NALT targeting phage have potential utility as a biological particulate mucosal adjuvant for stimulating an immune response to attached antigen(s).

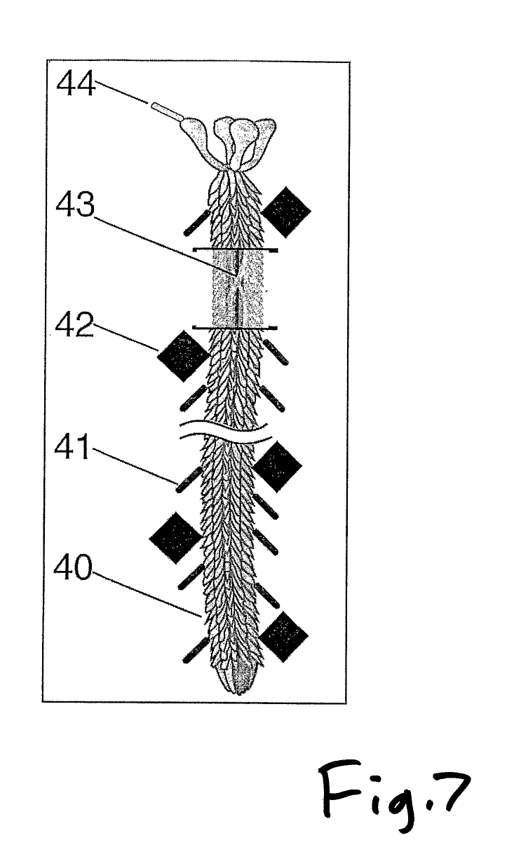

Accordingly, FIG. 7 illustrates a phage adjuvant 40, with additional immunostimulatory adjuvant element options, shown by callout numbers 43 and 44.

For example, an additional immunostimulatory element 43 can be a DNA sequence that contains known CpG motifs that can activate cell surface receptors such as the Toll receptor 9 (TRL9), with the CpG motifs being selected from various DNA sequences that have been reported in the literature as having an ability to stimulate an immune response. The TRL9 class of receptors is activated by bacterial and viral DNA that is rich in CpG motifs, as reviewed in Krieg 2002, and this stimulates and robust immune response (Chu et al 1997). Klinman et al 2004 also provides a review of CpG motifs and their properties. Briefly, that review describes three primary classes of CpG sequences with immune stimulation properties, which have been designated as D-type, K-type, and C-type. D-type sequences trigger maturation of antigen presenting cells and directly stimulate interferon (IFN) from pDCs. K-type sequences activate pDCs and trigger B cells to proliferate and secrete. C-type sequences combine properties of K-type and D-type sequences in that they can directly stimulate B cells to secrete interleukin-6 (IL-6) and pDCs to produce IFN.

In a preferred example, a sequence based on a D-type sequence is inserted into the sequence for NALT targeting phage so as to not interfere with the normal production and coat proteins of the phage. By appropriately inserting the D-type sequence D-19 (described in Gursel et al 2002) into NALT targeting phage and coupling antigen to it, the resulting complex can be forecast to stimulate monocytes to transform into dendritic cells for antigen presentation, thereby promoting a stronger immune response in humans.

In a preferred example, a sequence based on a C-type ODN is inserted into the sequence for NALT targeting phage so as to not interfere with the normal production and coat proteins of the phage. A C-type sequence described in Klinman et al 2004 may be used to stimulate pDC and B cells and induce production of IL-6 and IFN to stimulate the immune system.

In another preferred example, sequence CpG ODN 1826 can be used to stimulate a B cell mediated immune response in mice. Addition of CpG 1826 to Incomplete Freund's Adjuvant stimulated a Th1 immunity response that was greater than that stimulated by using Complete Freund's Adjuvant (Chu et al 1997). Thus, to address the problem of alternatives to Complete Freund's Adjuvant, it can be predicted that addition of NALT targeting phage DNA with immunostimulatory CpG 1826 motif insert should safely enhance the immunostimulatory properties of Incomplete Freund's Adjuvant to a level equivalent to that of Complete Freund's Adjuvant and avoid exposure to the mycobacterium M. tuberculosis (or M. bovis). CpG 1826 has been used to facilitate the immune response of mice receiving radiotherapy treatment for a fibrosarcoma tumor (Manson et al., 2005).

In one preferred example, sequence CpG 7909 can be used to stimulate an immune response in humans. CpG 7909 has been optimized to stimulate human plasmacytoid DCs (pDCs) and B cells in vitro and in vivo (Kreig, 2002) and has been used clinically with Incomplete Freund's Adjuvant to stimulate a strong immune response to a tumour antigen (Speiser et al., 2005). Co-injection of CpG 7909 with antigen also stimulates production of higher affinity IgG antibodies (Siegrist et al., 2004).

It is important to note that this invention presents a significant advance on the field working with CpG motifs presented to immune system as synthetic oligonucleotides (ODNs). Filamentous phage provide a fundamentally different technology platform for developing and delivering CpG sequences as immune adjuvants. ODNs are typically synthesized to be resistant to DNAase activity. In contrast, an immunostimulatory CpG insert into phage DNA is shielded from DNAase inactivation by the phage coat proteins which are removed within the target cell during processing and exposed to the intracellular TLR9 receptor. In contrast to ODNs, which become increasingly expensive to manufacture and purify as the number of base pairs in the oligonucleotide increases, the size of the filamentous phage DNA is not size constrained (longer DNA sequence results in longer phage particle). That is, it is practical to insert immunostimulatory repeating CpG sequences of even more than +200 bp length and so experiment with a larger number of CpG permutations, for example, combining CpG sequences for rodent and non-rodent species. Further, the field has had to make use of methods such as liposome vesicles or other means of linkage the antigen to the ODN (eg Mizu et al., 2004) to obtain benefit of the CpG ODN immunostimulatory adjuvant. Because bacteriophage DNA with CpG motif inserts is naturally encapsulated by phage coat proteins, it can be a simple matter to couple the antigen to phage by chemical crosslinking (such as can be achieved by using glutaraldehyde).

Antigen may be coupled directly to NALT targeting phage with CpG motif inserts by chemical means (eg glutaraldehyde or chemical crosslinker such as described in Tani et al 2005) or molecular biological means (by appropriately inserting the gene encoding the antigen into a gene encoding a coat protein for the NALT targeting peptide display phage). Phage have been used to display antigens for immunisation purposes since the mid 1990s (De Berardinis et al 2003, reviewed in Perham et al 1995).

If desired, other stimulators of an immune response may be inserted to potentiate an immune response to the particulate adjuvant. FIG. 7 depicts additional immunostimulatory adjuvant elements 44. One example of an additional immunostimulatory element is a ligand for Toll receptor 5 such as recombinant flagellin that may be prepared by referring to methods and references in Ramos et al 2004 and engineered into phage DNA for expression on coat protein III.

It should be noted that with the exception of TRL9, most TRL receptors including Toll receptor 5 are expressed by a wide range of tissues and hence many TRL ligands can stimulate a systemic response that may be toxic. In contrast, in humans TRL9 is expressed only by plasmacytoid dendritic cells and B cells and so presents a much lower risk of systemic toxicity (Hemmi et al, 2000). This is important where it is intended that the adjuvant be used with the highest level of safety for the subject.

Intranasally administered, the NALT targeting adjuvant-antigen complex will be efficiently delivered to the NALT to stimulate a desired immune response. Examples of antigens include any of a wide range of pathogens including anthrax (eg see Boyaka et al., 2003), influenza (eg see Joseph et al., 2002), rabies, polio, etc. Issues to be addressed in the pre-clinical and clinical studies of the nasal adjuvant are described in CDER (2003). A number of these issues relate to the passage of the adjuvant into the brain. Methods disclosed in this invention may be used in addressing such issues. Because of the biological nature of the adjuvant, very large amounts of the adjuvant-antigen complex may be prepared relatively inexpensively for low cost mass immunization such as in the developing world.

### REFERENCES

Agdeppa E D, Kepe V, Lui J, Flores-Torres S, Satyamurthy N, Petric A, Cole G M, Small G W, Huang S C, Parrio J R (2001) Binding characteristics of radiofluorinated 6-dialkylamino-2-naphthylethylidene derivatives as positron emission tomography imaging probes for beta-amyloid plaques in Alzheimer's disease. J Neurosci 21:RC189.
Asanuma H, Thompson A H, Iwasaki T, Sato Y, Inaba Y, Aizawa C, Kurata T, and Tamura S (1997) Isolation and characterisation of mouse nasal-associated lymphoid tissue. J Immunol Methods 202: 123-131.
Baker A and Cotton M (1997) Nucl Acids Res 25: 1950-1956
Baker H and Spencer R F (1986) Transneuronal transport of peroxidase-conjugated wheat germ agglutinin (WGA-HRP) from the olfactory epithelium to the brain of the adult rat. Exp. Brain Res. 63(3): 461-73.
Balin B J, Broadwell R D, Salcman M, el-Kalliny M. (1986) Avenues for entry of peripherally administered protein to the central nervous system in mouse, rat, and squirrel monkey. J Comp Neurol. 251: 260-80.
Bax B D V, Blundell T L, Murray-Rust J, McDonald NQ (1997) Crystal structure of 7S NGF: A complex of nerve growth factor with four serine binding proteins (serine proteases). MMDB Id: 7621 PDB Id: ISGF
Behl C R, Pimplaskar H K, Sileno A P, deMeireles J, Romeo V D (1998) Effects of physiochemical properties and other factors on systemic nasal drug delivery. Adv. Drug Del Rev 29: 89-116.
Berg L, McKeel D W Jr, Miller J P, Storandt M, Rubin E H, Morris J C, Baty J, Coats M, Norton J, Goate A M, Price J L, Gearing M, Mirra S S, Saunders A M (1998) Clinicopathologic studies in cognitively healthy aging and Alzheimer's disease: relation of histological markers to dementia severity, age, sex and apolipoprotein E genotype. Arch Neurol 55: 326-335.
Braak H, Del Tredici K, Rub U, de Vos R A I, Jansen Steur E N H, Braak E (2003) Staging of pathology related to sporadic Parkinson's disease. Neurobiol Aging 24: 197-211.
Braak H, Ghebremedhin E, Rub U, Bratzke H, Del Tredici K (2004) Stages in the development of Parkinson's disease-related pathology. Cell Tissue Res 318: 121-134.
Broadwell R D and Balin B L (1985) Endocytic and exocytic pathways of the neuronal secretory process and trans-synaptic transfer of wheat germ agglutinin-horseradish peroxidase in vivo. J. Comp. Neurol 242: 632-650.
Born J, Lange T, Kern W, McGregor G P, Bickel U, Fehm H L. (2002) Sniffing neuropeptides: a transnasal approach to the human brain. Nature Neurosci 5: 514-516.
Brown A (2003) Axonal transport of membranous and nonmembranous cargoes: a unified perspective. J Cell Biol. 160: 817-21
Capsoni S, Ugolini G, Comparini A, Ruberti F, Berardi N, Cattaneo A (2000) Alzheimer-like neurodegeneration in aged anti-nerve growth factor transgenic mice. Proc Natl Acad Sci USA 97: 6826-6831.
Capsoni S, Giannotta S, Cattaneo A. (2002) Nerve growth factor and galantamine ameliorate early signs of neurodegeneration in anti-nerve growth factor mice. PNAS 99: 12432-7
Carrera M R A, Kaufmann G F, Mee J M, Meijler M M, Koob G F, and Janda K D (2004) Treating cocaine addition with viruses. Proc Natl Acad Sci USA 101: 10416-10421
Chandler C E, Parsons L M, Hosang M, Shooter E M (1984) A monoclonal antibody modulates the interaction of nerve growth factor with PC12 cells. J Biol Chem. 259: 6882-9.
Chen X-Q, Fawcett J R, Rahman Y-E, Ala T A, Frey W H (1998) Delivery of nerve growth factor to the brain via the olfactory pathway. J Alzheimer Dis 1: 35-44.
Crutcher K A, Scott S A, Liang S, Everson W V, Weingartner J (1993) Detection of NGF-like activity in human brain tissue: increased levels in Alzheimer's disease. J Neurosci. 13: 2540-2550.
Dabrowska K, Switala-Jjelen K, Opolski A, Weber-Dabroska and Gorski A (2004) Bacteriophage penetration in vertebrates û A review. J Applied Microbiol 98: 7-13.
De Lacalle S, Lim C, Sobreviela T, et al. (1994) J. Comp Neurol. 345: 321-344
DeKosky S T and Marek K (2003) Looking backwards to move forwards: Early detection of neurodegenerative disorders. Science. 302: 830-834.
DeKosky S T, Ikonovovic M D, Styren S D, Beckett L, Wisniewski S, Bennett D A, Kordower J H, E J Mufson (2002) Upregulation of choline acetyltransferase activity in hippocampus and frontal cortex of elderly subjects with mild cognitive impairment. Ann Neurol 51: 145.
DiStefano P S, Johnson E M Jr. (1988) Identification of a truncated form of the nerve growth factor receptor. PNAS 85: 270-4
Edman P and Bjork E (1992) Nasal delivery of peptide drugs. Adv Drug Deliv Rev 8: 165-177.
Essler M and Ruoslahti E (2002) Proc Natl Acad Sci USA 99: 2252-2257
Fehm H L, Smolnik R, Kern W, McGregor G P, Bickel U, Born J (2001) The melanocortin melanocyte-stimulating hormone-adrenocorticotropin 4-10 decreases body fat in humans. J. Clinic. Endrocrinol. Metabol. 86: 1144-1148.
Ferguson I A, Schweitzer J B, Bartlett P F, Johnson E M Jr (1991) Receptor-mediated retrograde transport in CNS neurons after intraventricular administration of NGF and growth factors. J Comp Neurol. 313: 680-92.
Fischer W, Wictorin K, Bjorklund A, et al. (1988) Nature 329: 65-68.
Frenkel D, Solomon B (2002) Filamentous phage as vector-mediated antibody delivery to the brain. PNAS 99: 5675-9
Frey W H Liu T Thorne R G Rahman Y-E (1995) Intranasal delivery of 125I-labelled nerve growth factor into the brain via the olfactory route. In: Iqbal K, Mortimer J A, Windbland B, Wiseniewski H M (eds) Research Advances in Alzheimer's Disease and Related Disorders. New York, N.Y.; John Wiley & Sons Ltd pp 329-335.
Frey W H, Liu J, Chen X, Thorne R G, Fawcett J R, Ala T A et al (1997) Delivery of 125I-NGF to the brain via the olfactory route. Drug Delivery. 4: 87-92.
Frey W H II (2002) Bypassing the blood-brain barrier to delivery therapeutic agents to the brain and spinal cord. http://www.drugdeliverytech.com/cgi-bin/articles.cgi?idArticle=61
Gold B G, Densmore V, Shou W, Matzuk M M, Gordon H S (1999) Immunophilin FK506-binding protein 52 (not FK506-binding protein 12) mediates the neurotrophic action of FK506. J Pharmacol Exp Ther 289: 1202-1210.
Government Guideline (2003) Practice parameter: early diagnosis of dementia: mild cognitive impairment (an evidence-based review). Report of the Quality Standards Subcommittee of the American Academy of Neurology. NGC summary, www.guidelines.gov/summary/summary.aspx?doc_id=2816&nnbr=2042&string=% 22Alzheim*%22.
Goldenberg and Sharkey, 1993
Gozes I, Bardea A, Reshef A, Zamoostiano R, Zhukovsky S, Rubinraut S et al (1996) Neuroprotective strategy for Alzheimer disease: Intranasal administration of fatty neuropeptide. Proc Natl Acad Sci USA 93: 91-94.
Gozens I, Giladi E, Pinhasov A, Bardea A, Brenneman D E (2000) Activity dependent neurotrophic factor: intranasal administration of femtomolar-acting peptides improve performance in a water maze. J Pharmacol Exp Ther 293: 1091-1098.
Gustafsson E, Andsberg G, Darsalia V, et al. (2003) Eur J Neurosci 17: 2667-2678.
Hefti F, Dravide A, Hartikka J (1984) Brain Res 293: 305-309.
Higgins L M, Lambkin I, Doonelly G, Byrne D, Wilson C, Dee J, Smith M, and O'Mahony D J (2004) In vivo phage display to identify M cell-targeting ligands. Pharm Res 21: 695-705.
Holland D R, Cousenes L S, Meng W, and Matthews B W (1994) nerve growth factor in different crystal forms display structural flexibility and reveal zinc binding sites. J Mol Biol 239: 385-400.
Holland D R and Matthews B W (1995) 1BTG; Nerve Growth Factor; X-ray Diffraction; 2.50 A, www.rcsb.org/pdb/cgi/
Ibanez C F (1995) Neurotrophic factors: from structure-function studies to designing effective therapeutics. Trends Biotechnol. 13: 217-227.
Levine M M (2003) Can needle-free administration of vaccines become the norm in global immunization? Nature Medicine 9: 99-103.
Illum L (2000) Transport of drugs from the nasal cavity to the central nervous system. Eur J Pharm Sci 11: 1-18.
Illum L (2002) Nasal drug delivery: new developments and strategies. Drug Discovery Today 7: 1184-9. Review.
Kern W, Schieder B, Schwarzenburg J, Strange E F, Born J, Fehm H L (1997) Evidence for central nervous effects of corticotropin-releasing hormone on gastric acid secretion in humans. Clin Neuroendocrinol 65: 291-298.
Kern W, Born J, Schreiber H, Fehm H L (1999) Central nervous system effects of intranasally administered insulin during euglycemia in men. Diabetes. 48: 557-563.
Kipriyanov S M, Le Gall F. (2004) Recent advances in the generation of bispecific antibodies for tumor immunotherapy. Curr Opin Drug Discov Devel. 7: 233-42.
Klockether T (2004) Parkinson's disease: clinical aspects. Cell Tissue Res 318: 115-120.
Klunk W E, Engler H, Nordberg A, Bacskai B J, Wang Y, Price J C, Bergstrom M, Hyman B T, Langstrom B, Mathis C A (2003) Imaging the pathology of Alzheimer's disease: amyloid-imaging with positron emission tomography. Neuroimaging Clin N Am 13: 781-789.
Knopman D S, DeKosky S T, Cummings J L, Chui H, Corey-Bloom J, Relkin N, Small G W, Miller B, Stevens J C (2001) Practice parameter: diagnosis of dementia. Neurology 56: 1143-1153.
Kontermann R E (2005) Recombinant bispecific antibodies for cancer therapy. Acta Pharmacol Sin. 26: 1-9.
Kucheryanu V G, Kryzhanovsky G N, Kudrin V S, Yurasov V V, Zhigaltev I V, Bocharov E V (1999) Intranasal fibroblast growth factors: delivery into the brain exerts antiparkinsonian effect in mice. In: V Torchilin, Veronese F M eds. Proc 26th International Symposium on Controlled Release of Bioactive Materials. Boston Mass.: Controlled Release Society Inc pp 643-644.
Kufer P, Lutterbuse R, Baeuerle P A. (2004) A revival of bispecific antibodies. Trends Biotechnol. 22: 238-44.
Kuper C F, Koornstra P J, Hameleers D M H, Biewnga J, Spit B J, Duijvestinjn A M, Vriesman P J C and Sminia T (1992) The role of nasopharyngeal lymphoid tissue. Immunol Today 13: 219-224.
Kung H F (1991) Overview of radiopharmaceuticals for diagnosis of central nervous system disorders. Crit Rev Clin Lab Sci 28: 269-286.
Liu X F, Fawcett J R, Thorne R G, Frey W H (2001a) Non-invasive intranasal insulin-like growth factor-I reduces infarct volume and improves neurological function in rats following middle cerebral artery occlusion. Neurosci Lett 308: 91-94.
Liu X F, Fawcett J R, Thorne R G, DeFor T A, Frey W H (2001b) Intranasal administration of insulin-like growth factor-I bypasses the blood brain barrier and protects against focal cerebral ischemic damage. J Neurol Sci 187: 91-97.
Loewnstein D A, Ownby R, Scram L, Acevedo A, Rubert M, Arguelles T (2001) An evaluation of the NINCDS-ADRDA neuropsychological criteria for the assessment of Alzheimer's disease: a confirmatory factor analysis of single versus multi-factor models. J Clin Exp Neuropsychol 23: 274-284.
McDonald N Q, Lapatto R, Murray-Rust J, Gunning J, Wlodawer A, Blundell T L (1993) 1BET; Nerve Growth Factor; X-ray-Diffraction; 2.30 A www.rcsb.org/pcsb/
Man A L, Prieto-Garcia M E and Nicoletti C (2004) Improving M cell mediated transport across mucosal barriers: do certain bacteria hold the key. Immunol 113: 15-22.
Massa S M, Xie Y and Longo F M (2003) Alzheimer's therapeutics: neurotrophin domain small molecule mimetics. J Mol Neurosci 20: 323-326.
Mathis C A, Klunk W E, Price J C, DeKosky S T (2005) Imaging technology for neurodegenerative diseases: progress towards detection of specific pathologies. Arch Neurol 62: 196-200.
Mathis C A, Wang Y, Klunk W E (2004) Imaging beta-amyloid plaques and neurofibrillary tangles in the aging human brain. Curr Phar Des 10: 1469-1492.
Mathison S, Nagilla R, Kompella U B (1998) Nasal route for direct delivery of solutes to the central nervous system: fact or fiction? J Drug Target 5: 415-441.
Moller J C, Kruttgen A, Heymach J V J, et al. (1998) J Neurosci Res 51: 463-472.
Morris J C, Storandt M, Miller J P, Mckeel D W, Price J L, Rubin E H, Berg L (2001) Mild cognitive impairment represents early stage-Alzheimer's disease. Arch Neurol 58: 397-405.
Mufson E J, Krowder J H (1999) Nerve growth factor in Alzheimer's disease. In: Peter A A, Morrison J H, ed. Cerebral cortex. New York: Kluwer Academic/Plenum Press. Pp 681-731.
Mufson E J, Ma S Y, Dills J, Chochran E J, Leurgans S, Wuu J, Bennett D A, Jaffar S, Gilmore M L, Levey A I, Kordower J H (2002) Loss of basal forebrain p75NTR immunoreactivity in subjects with mild cognitive impairment and Alzheimer's disease. J Comp Neurol 443: 136-153.
Muller W E, Stoll L, Schubert T, et al. (1991) Acta Psychiatr Scand Suppl 366: 34-9.
O'Hagan D T, MacKichan M L, Singh M (2001) Biomolecular Engineering 18: 69-85.
Pasqualini R and Ruoslahiti E (1996) Nature 380: 364-366; Essler M and Ruoslahti E (2002) Proc Natl Acad Sci USA 99: 2252-2257
Pautler R G (2004) In vivo, trans-synaptic tract-tracing utilising manganese-enhanced magnetic resonance imaging (MEMRI). NMR Biomed 17: 595-601.
Perras B, Marshall L, Kohler G, Born J, Fehm H L (1999) Sleep and endocrine changes after intranasal administration of growth hormone-releasing hormone in young and aged humans. Psychoneuroendocrinol 24: 743-756.
Petersen R C, Stevens J C, Ganguli M, Tangalos E G Cummings J L, DeKosky S T (2001) Practice parameter: Early detection of dementia: Mild cognitive impairment. Neurology 56: 1133-1142.
Pietrowsky R, Struben C, Molie M, Fehm H L, Born J (1996a) Brain potential changes after intranasal vs intravenous administration of vasopressin: evidence for a direct nose-brain pathway for peptide effects in humans. Biol Psychiatry. 39: 332-340.
Pietrowsky R, Thiemann A, Kern W, Fehm H L, Morne J (1996b) A nose-brain pathway for psychotropic peptides: evidence from a brain evoked potential study with cholecystokinin. Psychoneuroendocrinol. 21: 559-572.
Pollack S J and Harper S J (2002) Small molecule Trk receptor agonists and other neurotrophic factor mimetics. Curr Drug Targets CNS Neurol Disord 1: 59-80.
Ramirez J J, Caldwell J L, Majure M, et al. (2003) J Neurosci 23: 2797-2803.
Ramon G (1924) Ann Inst Pasteur 38: 1.
Reed B R and Jagust W J (1999) Opening a window on cerebral cholinergic function: PET imaging of acetylecholinesterase. Neurology 52: 680-682.
Scott S A, Mufson E J, Weingartner J A, Skau K A, Crutcher K A (1995) Nerve growth factor in Alzheimer's disease: increased levels throughout the brain coupled with declines in nucleus basalis. J Neurosci 15: 6213-6221.
Shipley M T (1985) Transport of molecules from nose to brain: transneuronal anterograde and retrograde labelling in the rat olfactory system by wheat germ agglutinin-horse radish peroxidase applied to the nasal epithelium. Brain Res Bull 15: 129-142.
Smith G P (1991) Surface presentation of protein epitopes using bacteriophage expression systems. Curr Opin Biotechnol. 2: 668-73.
Smith D E, Roberts J, Gage F H, et al. (1999) Proc Natl Acad Sci USA 96: 10893-10898.
Smolnik R, Molle M, Fehm H L, Born J (1999) Brain potentials and attention after acute subchronic intranasal administration of ACTH4-10 and desacetyl-a-MSH in humans. Neuroendocrinol. 70: 63-72.
Sommer U, Hummel T, Cormann K, Mueller A, Frasnelli J, Kropp J, and Reichmann H (2004) Detection of presymptomatic Parkinson's disease: Combining smell tests, transcranial sonography and SPECT. Movement Disorders. 19: 1196-1202.
Steiner J P, Hamilton G S, Ross D T et al (1997) Neurotrophic immunophilin ligands stimulate structural and functional recovery in neurodegenerative animal models. Proc Natl Acad Sci USA 94: 2019-2024.
Stiansny-Kolster K, Doerr Y, Moller J C, Hoffken H, Behr T M, Oetel W H, Mayer G (2005) Combination of idiopathic REM sleep behaviour disorder and olfactory dysfunction as possible indicated for synucleinopathy demonstrated by dopamine transported FP-CIT-SPECT. Brain 128: 126-137.
Stokin G B, Lillo C, Falzone T L, Brusch R G, Rockenstein E, Mount S L, Raman R, Davies P, Masliah E, Williams D S, Goldstein L S B (2005) Axonopathy and transport deficits early in the pathogenesis of Alzheimer's disease. Science 307: 1282-1288.
Stroh M, Zipfel W R, Williams R M, Ma S C, Webb W W, Saltzman W M (2004) Multiphoton microscopy guides neurotrophin modification with poly(ethylene glycol) to enhance interstitial diffusion. Nature Materials 3: 489-494.
Tani H, Rush R A and Ferguson I A (2002) A novel in vivo selection procedure for isolating neuron internalising antibodies from a phage display library. 32th Annual meeting of Society for Neuroscience, Orlando, November 2-7. Proceedings 28: #609.16 9
Taniuchi M, Schweitzer J B, Johnson E M Jr (1986) Nerve growth factor receptor molecules in rat brain. Proc Natl Acad Sci 94: 2019-2024.
Thorne R G, Emory C R, Ala T A, Frey W H 2nd. (1995) Quantitative analysis of the olfactory pathway for drug delivery to the brain. Brain Res. 692: 278-82.
Thorne R G, Pronk G, Frey W H (2000) Delivery of insulin-like growth factor-1 to the brain and spinal cord along olfactory and trigeminal pathways following intranasal administration: a noninvasive method for bypassing the blood-brain barrier. Soc Neurosci. Abstract 26: 1365.
Thorne R G and Frey W H II (2001) Delivery of neurotrophic factors into the central nervous system. Clinical Pharmacokinetics 40: 907-946.
Tinsley R B, Vesey M J, Barati S et al. (2004) J Gene Med. 6: 1023-1032.
Tremere L A, Pinaud R, Grosche J, Hartig W, Rasmusson D D. (2000) Antibody for human p75 LNTR identifies cholinergic basal forebrain of non-primate species. Neuroreport. 11: 2177-83.
Ugwoke M I, Verbeke N and Kinget R (2001) The biopharmaceutical aspects of nasal mucoadhesive drug delivery. J Pharmacy and Pharmacol 53: 3-22.
Ultsch M H, Wiesmann C, Simmons L C, Henrich J, Yang M, Reilly D, Bass S H, de Vos A M (1999) Crystal structures of the neurotrophin-binding domain of TrkA, TrkB and TrkC. J Mol Biol 290: 149-159.
Van der Zee C E, Lourenssen S, Stanisz J and Diamond J (1995) Eur J Neurosci 7: 160-168.
Vaughan T J, Osbourn J K, Tempest P R. (1998) Human antibodies by design. Nat Biotechnol. 16: 535-9.
Van Ginkel F W, Jackson R J, Yuki Y and McKhee J R (2000) Cutting edge: the mucosal adjuvant cholera toxin redirects vaccine proteins into olfactory tissues. J Immunol 165: 4778-4782.
von Itzstein M, Wu W-Y, Kok G B et al (1993) Rational design of potent sialidase-based inhibitors of influenza virus replication. Nature 363: 401-402.
Wiesmann C, Ultsch M H, Bass S H, De Vos A M (1999) NGF binding domain of human trkA receptor MMDB Id: 10998 PDB Id: 1WWA
Wiesmann C, Ultsch M H, De Vos A M (1999) NGF in complex with domain 5 of the trkA receptor MMDB Id: 11178 PDB Id: 1WWW
Wu K, Meyers C A, Bennett J A, et al. (2004) Brain Res. 1008: 284-287.
Xie Y, Tisi M A, Yeo T T, Longo F M (2000) Nerve growth factor (NGF) loop 4 dimeric mimetics activated ERK and AKT and promote NGF-like neurotrophic effects. J Biol Chem 275: 29868-29874.
Xie Z, Guo N, Yu M, Hu M, Shen B. (2005) A new format of bispecific antibody: highly efficient heterodimerization, expression and tumor cell lysis. J Immunol Methods. 296(1-2):95-101.
Zupan A A and Johnson E M (1991) Evidence for endocytosis-dependent proteolysis in the generation of soluble truncated nerve growth factor receptors by A875 human melanoma cells. J Biol Chem. 266: 15384-90
Burggren A C, Bookheimer S Y (2002) Structural and functional neuroimaging in Alzheimer's disease: An update. Current Topics in Medicinal Chemistry 2: 385-393
Bush A I, “Metal complexing agents as therapies for Alzheimer's disease,” Neurobiol Aging. 2002 November-December; 23(6): 1031-8.
Lingford-Hughes A. Human brain imaging and substance abuse. Curr Opin Pharmacol. 2005 February; 5(1):42-6
Martin J B. Molecular basis of the neurodegenerative disorders. N Engl J Med 1999; 340: 1970-1980. [Erratum, N Engl J Med 1999; 341: 1407
Selkoe D J. Alzheimer's disease: genotypes, phenotypes, and treatments. Science 1997; 275: 630-631.
Walsh D M, Selkoe D J. (2004) Deciphering the molecular basis of memory failure in Alzheimer's disease. Neuron. 2004 Sep. 30; 44(1):181-93. Review.
Wevers A, Witter B, Moser N, Burghaus L, Banerjee C, Steinlein O K, Schutz U, de Vos R A, Steur E N, Lindstrom J, Schroder H. (2000) Classical Alzheimer features and cholinergic dysfunction: towards a unifying hypothesis? Acta Neurol Scand Suppl. 176: 42-8.

1. A method for evaluating the health of central nervous system neurons in a human patient, comprising:
a. administering to said patient a population of molecular complexes comprising:
(i) a polypeptide capable of activating neuronal endocytosis, axonal transport, and synaptic transfer, and
(ii) an imaging agent suited for determining location and evaluating neuronal transport of said molecular complexes within said patient; and
b. imaging said molecular complexes to determine location and evaluate neuronal transport of said molecular complexes within said patient.

2. The method of claim 1, wherein the polypeptide is a P75 receptor ligand.

3. The method of claim 2, wherein the P75 receptor ligand is an antibody specific to P75.

4. The method of claim 2, wherein the P75 receptor ligand is neurotrophin-3 (NT-3).

5. The method of claim 1, wherein the imaging of step (b) is conducted through fluorescent microscopy or single photon emission computerized tomography (SPECT).

6. The method of claim 1, wherein the imaging agent comprises ¹²³I.
